(19) United States
(12) Patent Application Publication
(10) Pub. No.: US 2010/0285164 A1
(43) Pub. Date: Nov. 11, 2010

(54) Orally Disintegrating Excipient

(75) Inventors: David Schaible, Ulster Park, NY (US); Louis Mejias, Hopewell Junction, NY (US)

Correspondence Address: Davidson, Davidson & Kappel, LLC, 485 7th Avenue, 14th Floor, New York, NY, 10018, US

(73) Assignee: JRS Pharma, Patterson, NY (US)

(21) Appl. No.: 12/464,073

(22) Filed: May 11, 2009

(51) Int. Cl. A61K 36/00; A61K 47/02; A61K 47/04; A61K 47/26; A61P 3/10; A61P 9/10; A61P 9/12; A61P 11/06; A61P 25/18; A61P 25/24

(52) U.S. Cl. 424/777; 514/771; 514/770; 514/781

(57) ABSTRACT

The present invention is directed to coprocessed excipient particles comprising a cellulosic material such as microcrystalline cellulose in intimate association with silicon dioxide, a disintegrant and a polyol, sugar or a polyol/sugar blend. The excipient particles display good processing and are useful in prepared compressed solid dosage forms that exhibit rapid disintegration (less than about 60 seconds) when placed on the tongue or when tested according to USP disintegration testing, while still providing acceptable mouth feel.

### FIELD OF THE INVENTION

The present invention provides a mono-particulate, directly compressible, orally disintegrating tablet (“ODT”) and an excipient composition comprising a cellulose coprocessed with a silicon dioxide, a polyol/sugar blend and optionally a disintegrant that has a high dilution potential and will produce compacts that are robust with low friability.

### BACKGROUND OF THE INVENTION

Traditional oral solid dosage forms are widely utilized in the pharmaceutical arts. Under certain circumstances, oral solid dosage form may be considered undesirable. Where the oral solid dosage form is large, it may be difficult to swallow. Further, there are patients that have great difficulty or are not capable of swallowing dosage forms that are not large. Typical patient populations that have difficulty in swallowing conventional oral solid dosage forms include young children and, in certain situations, the elderly. In other settings, drinking fluids to facilitate swallowing of conventional oral solid dosage forms may be inconvenient. If the patient is unable or averse to swallowing the dosage form, lapses in therapy could occur. Lack of patient compliance is well appreciated as a major difficulty in pharmacotherapy.

Alternative dosage forms have been created in an attempt to provide more acceptable alternatives to patients that have difficulty or are unable to swallow conventional oral solid dosage forms. Chewable tablets do not require swallowing oral solid dosage forms, but in certain cases are best administered with fluid. Additionally, chewable tablets often have an unpleasant taste and an unacceptable gritty texture.

Oral liquids also do not require swallowing oral solid dosage forms, but can also provide an unacceptable unpleasant taste. An additional complication with liquids is the risk of not administering the proper volume of the formulation as the liquid can easily be spilled while administering, or the full volume is not swallowed.

A newer oral dosage form technology known as orally dissolving or rapidly disintegrating dosage forms offer an attractive solution to conventionally swallowed oral solid dosage forms. Orally dissolving tablet (“ODT”), technology has been available from drug delivery companies such as Cardinal Health (Zydis®) utilizing freeze drying technology, Ethypharm (Flashtab®), utilizing hot melt extrusion, Eurand (Advatab®) and CIMA (Durasolv®/Orasolv®).

More recently, SPI Pharma has announced its successful commercialization of Pharmaburst. U.S. Pat. No. 7,118,765 to SPI Pharma, Inc. describes a quick-dissolve matrix for solid dosage forms. This system is a co-processed polyol product. It is hailed as a success and yet it has clear limitations.

Polyols have never been recognized as being very compactable. Additionally, they are generally considered to have poor dilution potential, particularly for poorly compactable drugs. SPI has provided a cough/cold formulation using Pharmaburst in an amount of 75.075% of the formulation. SPI recommends the use of 50-80% Pharmaburst. SPI product information states that the impact of reducing the Pharmaburst may give a faster disintegrating tablet but it may appear to be more “gritty” to the taste.

Despite all of the work outlined above and elsewhere, to date a true “off-the-shelf” (i.e., a premixed excipient) product to maximize the dispersion of the soluble component of an ODT throughout a highly compactable silicified microcrystalline cellulose (“SMCC”) system has not been achieved.

### OBJECTS AND SUMMARY OF THE INVENTION

It is an object of the present invention to provide an “off the shelf” (e.g., pre-manufactured”) excipient that can be used in the preparation of ODT products.

It is a further object of the present invention to provide an off the shelf excipient that comprises silicified microcrystalline cellulose, at least one polyol and a sugar.

In further embodiments of the invention, it is not necessary to include a compressibility augmenting agent, e.g., a metal oxide in the excipient. In such embodiments therefore, the invention comprises a pharmaceutical excipient composition, comprising agglomerated particles of a cellulosic material, a polyol, a sugar, and a disintegrant.

In further embodiments, the invention comprises a pharmaceutical excipient composition, comprising agglomerated particles of a cellulosic material, a disintegrant, and either a polyol or a sugar.

In still further embodiments, the metal oxide, e.g., colloidal silicon dioxide is replaced in whole or impart by a surfactant, a highly polar compound, or a combination thereof.

It is another object of the present invention to provide an off the shelf excipient that allows for high dilution while still maintaining an acceptable smooth and acceptable creamy mouth feel without being unacceptably gritty.

It is a still further object of the invention to provide an excipient product that has many of the tableting attributes of SMCC, but with the mouth feel and application of polyols that are ideal for orally dissolving dosage forms.

It is a further object of the present invention to provide an ODT having sufficient hardness and low friability, yet capable of dissolving within a short period of time, e.g. about 30 seconds.

It is another object of the invention to provide pharmaceutical formulations comprising at least one pharmaceutically active agent in an ODT product.

The invention is also directed to methods of preparing an excipient using in preparing an ODT product comprising co-processing MCC with CSD and a polyol, a sugar or a combination thereof. In certain embodiments, the process involves spray-drying.

The invention is also directed to methods of preparing an excipient using in preparing an ODT product comprising co-processing MCC with CSD and a polyol, a sugar or a combination thereof. In certain embodiments, the process involves spray-drying.

In accordance with the above objects and others, the present invention is related in part to a pharmaceutical excipient composition, comprising a cellulosic material in intimate association with a compressibility augmenting agent selected from the group consisting of a metal oxide, a surfactant and a mixture of the foregoing, a polyol, a sugar, and a disintegrant.

The invention is further related in part to a pharmaceutical excipient composition, comprising agglomerated particles of agglomerated particles of a cellulosic material, a metal oxide, a polyol, a sugar, and a disintegrant.

The invention is further directed to an oral solid dosage from comprising a compressed mixture of an excipient comprising agglomerated particles of a cellulosic material, a compressibility augmenting agent, one or more polyols, one or more sugars, and a disintegrant, an effective amount of an active agent; and an optional sweetening agent and an optional flavoring agent, wherein the oral solid dosage form substantially disintegrates within about, e.g., 90 seconds when placed on the tongue of a patient.

In certain preferred embodiments, the cellulosic component and the metal oxide are agglomerated together such that they are in intimate association with each other prior to mixing with the polyol, sugar and disintegrant. In other preferred embodiments, the cellulosic component, the metal oxide and the disintegrant are agglomerated together such that they are in intimate association with each other prior to mixing with the polyol, sugar.

In certain preferred embodiments, the cellulosic material is microcrystalline cellulose. In further preferred embodiments, the compressibility augmenting agent is a metal oxide, a surfactant, a highly polar compound or a mixture of any of the foregoing. In preferred embodiments, the intimately associated particles of cellulosic component (e.g., microcrystalline cellulose) and an effective amount of a compressibility augmenting agent to provide suitable compressibility to the final excipient composition in accordance with the present invention. Generally, the intimately associated particles of cellulosic component and compressibility augmenting agent comprise up to about 20 percent compressibility augmenting agent, by weight.

In certain preferred embodiments, the excipient composition of the invention comprises from about 10 to about 40% cellulosic material and from about 1 to about 10% metal oxide.

In certain preferred embodiments, the compressibility augmenting agent is a fumed or colloidal metal oxide. In most preferred embodiments, the compressibility augmenting agent comprises colloidal silicon dioxide.

In certain preferred embodiments, the polyol is selected from the group consisting of sorbitol, mannitol, xylitol, erythritol, maltitol, lactitol, isomalt, and mixtures thereof.

In certain preferred embodiments, the sugar is selected from the group consisting of lactose, fructose, dextrose, sucrose, maltose, xylose, mannose, and mixtures thereof.

The ratio of the polyol component to the sugar component in the excipient composition according to the invention is from about 99.1:0.9 to about 0.9:99.1, by weight. In certain preferred embodiments, the ratio of the polyol component to the sugar component is from about 80:20 to about 20:80, or from about 60:40 to about 40:60, and preferably about 1:1.

In certain preferred embodiments, the disintegrant comprises from about 1 to about 20% of the excipient composition and is selected from the group consisting of corn starch, modified corn starch, potato starch, modified potato starch, pregelatinized starch, sodium starch glycolate, a cross-linked polyvinyl pyrrolidone, alginate, a cellulosic, an ion exchange resin, a natural gum, a modified natural gum, a synthetic gum, chitin, chitosan, clay, agar, a gas evolving disintegrant. In certain preferred embodiments, the excipient comprises from about 2 to about 10% disintegrant, or from about 1.5 to about 7.5% disintegrant, and in certain embodiments about 5% disintegrant.

In certain preferred embodiments, the agglomerated particles of the excipient composition in accordance with the present invention have a d₅₀ value of about 50-160 μm, and in certain embodiments preferably about 120 μm. In certain preferred embodiments, the agglomerated particles of the excipient composition in accordance with the present invention have a d₁₀ value of about 15-45 μm, and in certain embodiments preferably about 35 μm. In certain preferred embodiments, the agglomerated particles of the excipient composition in accordance with the present invention have a d₉₀ value of about 200-300 μm, and in certain embodiments preferably about from about 225-285 μm, or about 255 μm.

In embodiments of the present invention directed to an oral solid dosage form, such as an ODT formulation, the oral solid dosage form comprises from about 0.1 to about 20% of a pharmaceutically acceptable lubricant (e.g., for tableting), such as sodium stearyl fumarate. The amount of lubricant may be from about 0.5 to about 10%, or about 2%.

The oral solid dosage form also optionally, but preferably, comprises a sweetening agent, a flavoring agent, or both. The sweetening agent may be, e.g., aspartame, acesulfame potassium, sucralose, saccharin, saccharin sodium, xylitol and combinations thereof. For example, the sweetening agent in certain preferred embodiments may be from about 0.1 to about 1% aspartame, from about 0.2 to about 0.7%, or about 0.5% aspartame.

The flavoring agent may be fruit, mint(s), raspberry, licorice, orange, lemon, grapefruit, caramel, vanilla, cherry, grape, coffee, chocolate, tea flavors, other flavors known to those skilled in the art, or any combination thereof may be included.

In embodiments of the invention where the excipient composition is incorporated into a solid dosage form, the excipient may be compressed into a tablet along with the active ingredient, lubricant, and optional sweetener(s)/flavoring agent(s) and any other optional pharmaceutical excipients. Alternatively, the excipient composition may be placed into a capsule.

In certain preferred embodiments, the solid dosage form has a tablet hardness of about 2.67+/−0.46 kp achieved, e.g., from a compression force of about 3.68+/−0.06 kN, or a tablet hardness of about 3.4+/−0.36 kp achieved, e.g., from a compression force of about 5.03+/−0.14 kN, a tablet hardness of about 6.16+/−0.35 kp achieved, e.g., from a compression force of about 6.89+/−0.16 kN, or a tablet hardness of about 8.63+/−0.31 kp achieved, e.g., from a compression force of about 8.2+/−0.25 kN. In any event, it is preferred that the solid dosage forms of the present invention provide adequate cushioning such that the taste-mask coating remains substantially intact and substantially uncracked following tableting compression.

While it is acceptable for ODT solid dosage forms in accordance with the invention to disintegrate in vivo in about 90 seconds or less when placed on the tongue, it is preferred that the ODT formulation disintegrates in about 30 seconds or less when placed on the tongue. It is further preferred that the ODT solid dosage form produces minimal gritty sensation upon disintegration, and that where the active agent has an unpleasant taste, it is substantially masked. Thus, in certain preferred embodiments, the active agent may be coated with a taste-mask coating.

Solid dosage forms prepared in accordance with the present invention may comprise from about 0.1 to about 99% active agent, more preferably from about 0.1 to about 50% active agent, and in certain embodiments preferably from about 0.1 to about 30% active agent.

The invention is further directed to a process of making the excipient composition of the present invention, comprising: dry blending the polyol component and sugar component to create a dry blend, preparing an aqueous slurry comprising the cellulosic material, the metal oxide and the disintegrant, contacting the aqueous slurry with the dry blend to obtain dry excipient particles comprising the cellulosic material, the metal oxide, the disintegrant and the dry blend, and recovering the excipient.

In certain preferred embodiments, the aqueous slurry and dry blend of particles are contacted to each other in a spray dryer. The spray dryer may utilize a rotary atomizer, e.g., a two fluid nozzle atomizer. The dry blend may be introduced into the drying chamber through a single opening or multiple openings. The inlet temperature in the drying chamber may be from about 150 to about 275 degrees Celsius, or from about 175 to about 250 degrees Celsius, or about 220 degrees Celsius. The outlet temperature in the drying chamber may be from about 65 to about 125 degrees Celsius, or from about 75 to about 105 degrees Celsius, or about 90 degrees Celsius.

“Acceptable mouth feel upon disintegration” is understood to mean that the formulation does not cause excessive dryness in the mouth of a human patient and that an unacceptable gritty sensation is not sensed in the mouth of a human patient upon disintegration of the solid dosage form of the present invention.

### DETAILED DESCRIPTION OF THE INVENTION

The present invention is directed in part to a monoparticulate excipient comprising agglomerated particles comprising a cellulose component in intimate association with a polyol/sugar component, a silicon dioxide component and a disintegrant. In one aspect of the invention, the cellulose, metal oxide (e.g. silicon dioxide) and disintegrant are added to a solvent to create a cellulose, silicon dioxide and disintegrant slurry. In certain preferred embodiments, an aqueous solvent is utilized. The slurry is then preferably atomized and contacted with a dry blend polyol/sugar component in a drying environment, e.g., a drying chamber of a spray-drying apparatus. The solvent is then preferably removed, e.g., evaporated to provide the agglomerated excipient monoparticulates comprising a cellulose component, a silicon dioxide component, a disintegrant component, and a polyol/sugar component.

In certain embodiments, two are more components are in intimate association with each other. In certain embodiments, the cellulose, silicon dioxide and disintegrant are in intimate association. In still other embodiments, the cellulose, silicon dioxide and disintegrant and in intimate associate with the polyol and/or sugar.

In another aspect of the invention, the excipient particles are prepared by first preparing silicified microcrystalline cellulose agglomerated particles wherein the cellulose and the silicon dioxide are in intimate association. The agglomerated silicified microcrystalline cellulose particles are then processed as described herein with a disintegrant and a polyol/sugar blend to obtain the agglomerated particles of the present invention.

### FDA Guidance on Orally Dissolving Tablets

A Final Guidance for Industry for Orally Disintegrating Tablets, December 2008 released by Food and Drug Administration (“FDA”) recommends products labeled as ODTs should match the characteristics for this dosage form (i.e., rapid disintegration in saliva without need for chewing or drinking liquids). See: Center for Drug Evaluation and Research (CDER), Guidance for Industry. Orally Disintegrating Tablets. December 2008 (“FDA Guidance”); available on the FDA website at http://www.fda.gov/cder/Guidance/8528fnl.pdf. Based on the original product rationale and CDER experience, the guidance recommends that, in addition to the original definition, ODTs be considered solid oral preparations that disintegrate rapidly in the oral cavity, with an in vitro disintegration time of approximately 30 seconds or less, when based on the United States Pharmacopeia (USP 29 <701> Disintegration pp. 2670-2672, test method or alternatives that can be correlated with or are demonstrated to provide results equivalent to the USP method. FDA Guidance at pg. 3.

Although the value of 30 seconds is given as a desired result, it is not intended to represent an arbitrary distinction between an ODT and some other tablet form. It is instead representative of a general time period associated with drug products that have been found to have performance characteristics appropriate for a disintegrating tablet meant to be taken without chewing or liquids. FDA Guidance at pg. 3.

CDER recommends that as a primary consideration when developing this type of product, manufacturers should use the defining characteristics for this dosage form designation (rapid disintegration in saliva without need for chewing or liquids). FDA Guidance at pg. 3.

### Agglomerated Excipient Particles

The present invention is directed in part to a monoparticulate excipient comprising agglomerated particles comprising a cellulose component in intimate association with a polyol/sugar component, a silicon dioxide component and a disintegrant. In one aspect of the invention, the cellulose, metal oxide (e.g. silicon dioxide) and disintegrant are added to a solvent to create a cellulose, silicon dioxide and disintegrant slurry. In certain preferred embodiments, an aqueous solvent is utilized. The slurry is then atomized and contacted with a dry blend polyol/sugar component in a drying environment, e.g., a drying chamber of a spray-drying apparatus. The solvent is then preferably removed, e.g., evaporated to provide the agglomerated excipient monoparticulates comprising a cellulose component, a silicon dioxide component, a disintegrant component, and a polyol/sugar component.

In certain embodiments, two are more components are in intimate association with each other. In certain embodiments, the cellulose, silicon dioxide and disintegrant are in intimate association. In still other embodiments, the cellulose, silicon dioxide and disintegrant and in intimate associate with the polyol and/or sugar.

In another aspect of the invention, the excipient particles are prepared by first preparing silicified microcrystalline cellulose agglomerated particles wherein the cellulose and the silicon dioxide are in intimate association. The agglomerated silicified microcrystalline cellulose particles are then processed as described herein with a disintegrant and a polyol/sugar blend to obtain the agglomerated particles of the present invention.

Orally Dissolving Solid Dosage Forms Prepared with Premanufactured Agglomerated Excipient Particles

In another aspect of the present invention, the agglomerated monoparticulate excipient of the present invention is blended with an active agent and optionally other excipients, and compressed into a solid dosage form.

### Disintegration

In certain embodiments, when placed on the tongue or when tested under USP 29, <701> Disintegration test method, the solid dosage form of the present invention substantially disintegrates within about 3 minutes or less. In certain other embodiments, the solid dosage form substantially disintegrates within 90 seconds or less. In certain preferred embodiments, the solid dosage form substantially disintegrates in 60 seconds or less. In still further preferred embodiments, the solid dosage form disintegrates in about 30 seconds or less.

### Mouth Feel

In certain embodiments, the solid dosage formulations provide an acceptable mouth feel upon disintegration on the tongue or in the oral cavity. In certain embodiments, the solid dosage formulations of the present invention provide a sensation that is creamy and/or substantially without a gritty sensation upon disintegration when placed on the tongue, or in the oral cavity of a human patient. In certain embodiments, acceptable mouth feel is provided by the utilization of mannitol in the agglomerated particles.

### Taste

In certain other embodiments, the active agent has an unpleasant taste that is sufficiently masked during administration of the dosage form by the sensation provided by the polyol/sugar blend component when placed on the tongue, or in the oral cavity of a human patient. In other embodiments of the invention, additional agents described herein, e.g., sweeteners and/or flavoring agents are added to the formulation to provide taste masking. In still other embodiments, the active agent has an unpleasant taste that is sufficiently masked during administration of the dosage form. In certain embodiments, the active agent particles are coated with a film forming material as set forth in further detail herein. The coated active agent particles are then combined with the excipient composition set forth herein, and compressed into an orally dissolving tablet.

In certain other embodiments, an additional taste-masking agent is added to the blend prior to tableting or forming an orally dissolving solid dosage form. In still other embodiments, the excipient particles provide a cushioning effect substantially preventing the cracking of the taste-mask coating under compression during the tableting process.

### Process for Making the Agglomerated Excipient Particles

In certain embodiments, the present invention is also directed to processes for making agglomerated excipient particles. In certain preferred embodiments, the process involves preparing an aqueous slurry of a cellulose component, a metal oxide (e.g. silicon dioxide) component and a disintegrant component; atomizing the slurry, separately preparing a dry powder blend of a polyol and sugar; and contacting the dry powder blend with the atomized aqueous slurry in a drying chamber to form the agglomerated excipient particles of the invention.

In certain embodiments, as set forth in certain Examples described in detail below, the agglomerated particle excipients and orally disintegrating solid dosage forms of the present invention are prepared utilizing a premanufactured coprocessed silicified microcrystalline cellulose available as Prosolv® (available from JRS Pharma LP, Patterson, N.Y.). Processes for preparing silicified microcrystalline cellulose are described in U.S. Pat. No. 5,585,115, the disclosure of which is hereby incorporated by reference in its entirety. In certain embodiments of the invention, silicified microcrystalline cellulose, itself a coprocessed excipient comprising agglomerated particles of microcrystalline cellulose and colloidal silicon dioxde, is slurried with an aqueous or nonaqueous solvent and additional components, such as a disintegrant, are added into the slurry prior to contacting the atomized slurry with the dry powder polyol/sugar blend in a drying chamber to form the agglomerated particles.

In certain other preferred embodiments, the cellulose component and silicon dioxide) component are added separately to a solvent to prepare a slurry and additional components, such as a disintegrant, are added into the slurry prior to contacting the atomized slurry with the dry powder polyol/sugar blend in a drying chamber to form the agglomerated particles.

In certain other preferred embodiments, microcrystalline cellulose, colloidal silicon dioxide and crospovidone are mixed into an aqueous slurry; the slurry is atomized and contacted with a dry powder blend of polyol/sugar in a drying chamber. In certain preferred embodiments, the slurry is spray dried in a spray dryer while the dry powder polyol/sugar blend component is added to the drying chamber. In certain other embodiments, a disintegrant such as crospovidone XL is added to the slurry prior to atomizing.

In certain embodiments, the aforementioned aqueous slurry, with or without the additional ingredients of the final ODT excipient product, additional ingredients are dried to obtain the ODT excipient particles of the invention. Suitable means for drying the aforementioned aqueous dispersion include, but are not limited to spray drying and solvent evaporation. These drying means are exemplary and are not meant to be exclusive.

### Spray Drying

In the spray drying process, the aqueous dispersion of cellulose, e.g., microcrystalline cellulose and metal oxide, e.g, silicon dioxide, or, colloidal silicon dioxide (and in certain preferred embodiments other excipients, e.g., a disintegrant such as crospovidone) is brought together with a sufficient volume of hot air and preferably dry components such as a polyol/sugar dry blend to produce evaporation and drying of the liquid droplets of the dispersion. The highly dispersed slurry of (e.g., microcrystalline cellulose) and silicon dioxide is pumpable and capable of being atomized. It is sprayed into a current of warm filtered air, which supplies the heat for evaporation and conveys a dried product to a collecting device. The air is then exhausted with the removed moisture. In certain embodiments, the resultant spray-dried powder particles are approximately spherical in shape and are relatively uniform in size, thereby possessing excellent flowability. In certain embodiments, the agglomerated monoparticulate excipient product contains microcrystalline cellulose, silicon dioxide, a disintegrant and a polyol sugar blend in intimate association with each other.

In certain embodiments where the slurry contained microcrystalline cellulose and silicon dioxide, magnifications of the resultant particles indicate that the silicon dioxide is integrated with, or partially coats, the surfaces of the microcrystalline cellulose particles to form agglomerates. When the amount of silicon dioxide including in the excipient is greater than about 20% by weight relative to the microcrystalline cellulose, the silicon dioxide appears to substantially coat the surfaces of the microcrystalline cellulose particles. The exact relationship of the ingredients of the excipients after coprocessing is not presently understood; however, for purposes of description the coprocessed particles are described herein as including an agglomerate of microcrystalline cellulose and silicon dioxide (and optionally other components of the excipient), are in intimate association with each other.

### Intimate Association

By “intimate association”, it is meant that the silicon dioxide has in some manner been integrated with the microcrystalline cellulose particles, e.g., via a partial coating of the microcrystalline particles along with any additional ingredients included in the slurry or added as a dry powder contacting the atomized particles in the drying chamber, as opposed to a chemical interaction of the ingredients. The term “intimate association” is therefore deemed for purposes of the present description as being synonymous with “integrated” or “united”. The coprocessed agglomerated particles are not necessarily uniform or homogeneous. Rather, under magnification, e.g., scanning electron microscope at 500×, the silicon dioxide at the preferred percent inclusion appears to be an “edge-coating”. In certain preferred embodiments, all of the solid components used in the spray drying process are aggregated into aggregated (agglomerated) monoparticulates. In certain other preferred embodiments, these monoparticulates comprise microcrystalline cellulose, silicon dioxide, and crospovidone XL, which were included in an aqueous slurry. Mannitol and fructose were dry blended in a ratio of about 1:1 which was dry added into the drying chamber and contacted with the atomized slurry.

### Cellulose Component

All pharmaceutically acceptable cellulosic materials are contemplated by the invention including naturally occurring and modified celluloses, C1-6 alkycelluloses, hydroxy celluloses, hydroxy C1-6 alkyl celluloses and the like. One particularly preferred cellulosic component is microcrystalline cellulose (Emcocel® available from JRS Pharma LP, Patterson, N.Y.). Microcrystalline cellulose is a well-known tablet diluent and disintegrant. Its chief advantage over other excipients is that it can be directly compressed into self-binding tablets which disintegrate rapidly when placed into water. This widely-used ingredient is prepared by partially depolymerizing cellulose obtained as a pulp from fibrous plant material with dilute mineral acid solutions. Following hydrolysis, the hydrocellulose thereby obtained is purified via filtration and the aqueous slurry is spray dried to form dry, white odorless, tasteless crystalline powder of porous particles of a broad size distribution. Another method of preparing microcrystalline cellulose is disclosed in U.S. Pat. No. 3,141,875. This reference discloses subjecting cellulose to the hydrolytic action of hydrochloric acid at boiling temperatures so that amorphous cellulosic material can be removed and aggregates of crystalline cellulose are formed. The aggregates are collected by filtration, washed with water and aqueous ammonia and disintegrated into small fragments, often called cellulose crystallites by vigorous mechanical means such as a blender. Microcrystalline cellulose is commercially available in several grades which range in average particle size from 20 to 200 microns. For example, JRS Pharma offers air stream dried quality (Vivapur®) and spray dried quality (Emcocel®).

Microcrystalline cellulose is water-insoluble, but the material has the ability to draw fluid into a tablet by capillary action. The tablets then swell on contact and the microcrystalline cellulose thus acts as a disintegrating agent. The material has sufficient self-lubricating qualities so as to allow a lower level of lubricant as compared to other excipients.

Typically, microcrystalline cellulose has an apparent density of about 0.28 g/cm.sup.3 and a tap density of about 0.43 g/cm.sup.3. Handbook of Pharmaceutical Excipients, pages 53-55.

### Silicon Dioxide Component

Silicon dioxide is obtained by insolubilizing dissolved silica in sodium silicate solution. When obtained by the addition of sodium silicate to a mineral acid, the product is termed silica gel. When obtained by the destabilization of a solution of sodium silicate in such a manner as to yield very fine particles, the product is termed precipitated silica. Silicon dioxide is insoluble in water. Silicon dioxide, and in particular colloidal silicon dioxide, is used mainly as a glidant and anti-adherent in tabletting processes and encapsulation, promoting the flowability of the granulation. The amount of silicon dioxide included in such tablets for those applications is very limited, 0.1-0.5% by weight. Handbook of Pharmaceutical Excipients, © 1986 American Pharmaceutical Association, page 255. This is due in part to the fact that increasing the amount of silicon dioxide in the mixture to be tabletted causes the mixture to flow too well, causing a phenomena known to those skilled in the tabletting art as “flooding”. If the mixture flows too well, a varying tablet weight with uneven content uniformity can result.

All forms of silicon dioxide are contemplated for use in the present invention. In particular, silicon dioxide having an average primary particle size from about 1 nm to about 100 μm, and/or a surface area from about 10 m²/g to about 500 m²/g.

The silicon dioxide utilized in preferred embodiments of the invention is of the very fine particle size variety. In the most preferred embodiments of the invention, the silicon dioxide utilized is a colloidal silicon dioxide. Colloidal silicon dioxide is a submicron fumed silica prepared by the vapor-phase hydrolysis (e.g., at 1110 degrees Celsius) of a silicon compound, such as silicon tetrachloride. The product itself is a submicron, fluffy, light, loose, bluish-white, odorless and tasteless amorphous powder which is commercially available from a number of sources, including Cabot Corporation (under the tradename Cab-O-Sil); Degussa, Inc. (under the tradename Aerosil); E. I. DuPont & Co.; and W. R. Grace & Co. Colloidal silicon dioxide is also known as colloidal silica, fumed silica, light anhydrous silicic acid, silicic anhydride, and silicon dioxide fumed, among others. A variety of commercial grades of colloidal silicon dioxide are produced by varying the manufacturing process. These modifications do not affect the silica content, specific gravity, refractive index, color or amorphous form. However, these modifications are known to change the particle size, surface areas, and bulk densities of the colloidal silicon dioxide products.

The surface area of the preferred class of silicon dioxides utilized in the invention ranges from about 50 m²/gm to about 500 m²/gm. The average primary particle diameter of the preferred class of silicon dioxides utilized in the invention ranges from about 5 nm to about 50 nm. However, in commercial colloidal silicon dioxide products, these particles are agglomerated or aggregated to varying extents. The bulk density of the preferred class of silicon dioxides utilized in the invention ranges from about 20 g/l to about 100 g/l.

### Compressibility Augmenting Agent

In certain preferred embodiments, the cellulosic material (e.g., microcrystalline cellulose) and compressibility augmenting material are in intimate association with each other, prior to the introduction of other materials to form the excipient composition of the present invention. For purposes of the present invention, the amount of compressibility augmenting agent incorporated together in intimate association with the cellulosic material is generally described as an effective amount, i.e. an amount which enhances or augments the compressibility of the cellulosic material.

In preferred embodiments, the compressibility augmenting agent is selected from, e.g., a metal oxide, a surfactant, a highly polar compound, or mixtures of any of the foregoing.

Commercially available colloidal silicon dioxide products have, for example, a BET surface area ranging from about 50+/−15 m²/gm (Aerosil® OX50) to about 400+/−20 (Cab-O-Sil S-17) or 390+/−40 m²/gm (Cab-O-Sil EH-5). Commercially available particle sizes range from a nominal particle diameter of 7 nm (e.g., Cab-O-Sil S-17 or Cab-O-Sil EH-5) to an average primary particle size of 40 nm (Aerosil OX50). The density of these products range from 72.0+/−8 (Cab-O-Sil S-17) to 36.8 WI (e.g., Cab-O-Sil M-5). The pH of these products at 4% aqueous dispersion ranges from pH 3.5-4.5. These commercially available products are described for exemplification purposes of acceptable properties of the preferred class of silicon dioxides only, and this description is not meant to limit the scope of the invention in any manner whatsoever.

### Other Metal Oxides

One skilled in the art will appreciate that other classes of materials or compounds having size, surface area and other physical characteristics similar to those of silicon dioxide may be useful. Such materials include (but are not limited to) non-silicon metal oxides, preferably colloidal.

In certain preferred embodiments, the compressibility augmenting agent is a metal oxide.

In certain preferred embodiments of the invention, the additive material used is a fumed metal oxide, such as zirconium dioxide (ZrO₂), aluminum oxide (Al₂O₃) and titanium dioxide (TiO₂), as well as others, prepared by methods well known in the art.

### Surfactants

In certain preferred embodiments, the compressibility augmenting agent is a surfactant. The amount of surfactant coprocessed with the cellulosic material (e.g., microcrystalline cellulose) is dependent, in part, upon the type of surfactant selected. One particularly preferred surfactant is the anionic surfactant sodium lauryl sulfate (SLS). This surfactant is present in an amount of from about 0.1% to about 0.5% by weight of the cellulosic material. Preferably, however, the surfactant is present in amounts of from about 0.15 to about 0.4% and most preferably, in amounts ranging from about 0.2 to about 0.3% by weight.

In embodiments of the invention where the excipient composition is incorporated together with an active agent into a solid dosage form, such as an ODT formulation, the surfactant may be present in an amount of from about 0.1% to about 0.5% by weight based on the weight of the cellulosic material.

The surfactants which may be used in the present invention generally include all pharmaceutically-acceptable surfactants. Preferably, however, the surfactant is an ionic surfactant and most preferably, the surfactant is an anionic surfactant. Suitable pharmaceutically-acceptable anionic surfactants include, for example, those containing carboxylate, sulfonate, and sulfate ions. Those containing carboxylate ions are sometimes referred to as soaps and are generally prepared by saponification of natural fatty acid glycerides in alkaline solutions. The most common cations associated with these surfactants are sodium, potassium, ammonium and triethanolamine. The chain length of the fatty acids range from 12 to 18. Although a large number of alkyl sulfates are available as surfactants, one particularly preferred surfactant is sodium lauryl sulfate.

In the pharmaceutical arts, sodium lauryl sulfate has been used as an emulsifying agent in amounts of up to about 0.1% by weight of the formulation. It is not believed that surfactants such as SLS have been included in coprocessed MCC compositions. Moreover, it is not believed that surfactants have been used in the amounts described herein to improve the compressibility of MCC especially in wet granulations.

Sodium lauryl sulfate is a water-soluble salt, produced as a white or cream powder, crystals, or flakes and is used as a wetting agent and detergent. Also known as dodecyl sodium sulfate, SLS is actually a mixture of sodium alkyl sulfates consisting chiefly of sodium lauryl sulfate. Sodium lauryl sulfate is also known as sulfuric acid monododecyl ester sodium salt. Furthermore, sodium lauryl sulfate is readily available from commercial sources such as Sigma or Aldrich in both solid form and as a solution. The solubility of SLS is about 1 gm per 10 ml/water.

The fatty acids of coconut oil, consisting chiefly of lauric acid, are catalytically hydrogenated to form the corresponding alcohols. The alcohols are then esterified with sulfuric acid (sulfated) and the resulting mixture of alkyl bisulfates (alkyl sulfuric acids) is converted into sodium salts by reacting with alkali under controlled conditions of pH.

Alternative anionic surfactants include docusate salts such as the sodium salt thereof. Other suitable anionic surfactants include, without limitation, alkyl carboxylates, acyl lactylates, alkyl ether carboxylates, N-acyl sarcosinates, polyvalent alkyl carbonates, N-acyl glutamates, fatty acid, polypeptide condensates and sulfuric acid esters.

In other aspects of the invention amphoteric (amphipathic/amphiphilic surfactants), non-ionic surfactants and/or cationic surfactants are included in the coprocessed compositions of the invention. These alternative surfactants can be included to replace some or even all of the preferred anionic surfactant. It is preferred, however, that the surfactant comprise an anionic surfactant.

Suitable pharmaceutically-acceptable non-ionic surfactants such as, for example, polyoxyethylene compounds, lecithin, ethoxylated alcohols, ethoxylated esters, ethoxylated amides, polyoxypropylene compounds, propoxylated alcohols, ethoxylated/propoxylated block polymers, propoxylated esters, alkanolamides, amine oxides, fatty acid esters of polyhydric alcohols, ethylene glycol esters, diethylene glycol esters, propylene glycol esters, glycerol esters, polyglycerol fatty acid esters, SPAN's (e.g., sorbitan esters), TWEEN's (i.e., sucrose esters), glucose (dextrose) esters and simethicone.

Other suitable pharmaceutically-acceptable surfactants include acacia, benzalkonium chloride, cholesterol, emulsifying wax, glycerol monostearate, lanolin alcohols, lecithin, poloxamer, polyoxyethylene, and castor oil derivatives.

### Highly Polar Compounds

In yet other embodiments of the invention, the compressibility augmenting agent may be comprised of a highly polar compound. Examples of suitable a highly polar compounds include highly polar dyes, such as, for example, 3,3′-[[1,1′Biphenyl]-4,4′-diylbis-(azo)]bis[4-amino-1-naphthalenesulfonic acid]disodium salt; disodium salt of 6-hydroxy-5[(2-methyl-4-sulfophenyl)azo]-2-naphthalenesulfonic acid); 5-oxo-1-(p-sulfophenyl)-4-[(p-sulfophenyl)azo]-2-pyrazoline-3-carboxylic acid, trisodium salt), disodium salt of 1-p-sulphophenyl)azo-2-naphthol-6-sulfonic acid); trisodium-2-hydroxy-1-(4-sulfonato-1-naphthylazo)naphthalene-6,8-disulfonate); disodium 4,4′-(2,4-dihydroxy-5-hydroxymethyl-3,3-phenylene bisazo)di(napthalene-1-sulfonate)); tetrasodium 4-acetamido-5-hyroxy-6-[7-sulfonato-4-(4-sulfonatophenylazo)-1-naphthylazo]naphthalene-1,7-disulfonate); disodium 4-hydroxy-3-(4-sulfanato-1-naphythylazo) Naphthalene-1-sulfonate); trisodium 2-hydroxy-1-(4-sulfonato-1-naphthylazo)naphthalene-3,6-disulfonate); and mixtures thereof.

### Silicified Microcrystalline Cellulose

In certain embodiments, the cellulose component and the silicon dioxide component of the invention are first processed into premanufactured agglomerated particles. Agglomerated particles of the present invention may be prepared utilizing a premanufactured coprocessed silicified microcrystalline cellulose available as Prosolv® (available from JRS Pharma LP, Patterson, N.Y.). Processes for preparing silicified microcrystalline cellulose are described in U.S. Pat. No. 5,585,115, the disclosure of which is hereby incorporated by reference in its entirety. Prosolv is available in various grades including: Prosolv SMCC® 50 (60 μm average particle size measured by laser diffraction and bulk density 0.25-0.37 g/cm³); Prosolv SMCC® 50LM (equal quality to grade SMCC 50, but having a moisture content less than 3%); Prosolv SMCC® 90 (110 μm average particle size measured by laser diffraction and bulk density 0.25-0.37 g/cm³); Prosolv SMCC® 90LM (equal quality to grade SMCC 90, but having a moisture content less than 3%); Prosolv SMCC® HD90 (equal quality to grade SMCC 90LM, but having a bulk density 0.35-0.50 g/cm³); and Prosolv SMCC® HD90LM (equal quality to grade SMCC HD90, but having a moisture content less than 3%).

In alternative embodiments of the invention, microcrystalline cellulose and colloidal silicon dioxde are slurried with an aqueous or nonaqueous solvent, and additional components such as a disintegrant.

The aforementioned slurry is then atomized and contacted with the dry powder polyol, sugar or polyol/sugar blend component in a drying chamber to form the agglomerated particles of the present invention.

### Polyols

The polyols contemplated for use in the present invention include any pharmaceutically acceptable polyol. Non-limiting examples include mannitol, sorbitol, and xylitol. Mannitol, sorbitol, and xylitol are known by one of ordinary skill in the art to provide a cool creamy mouth feel upon dissolution in the oral cavity. The structures for mannitol, sorbitol and xylitol are set forth below in Table 1.

TABLE 1
Polyol Comparison

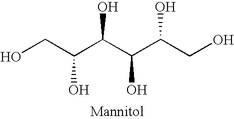

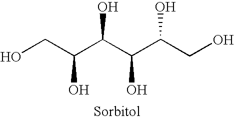

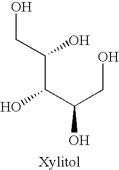

### Sugar Component

Sugars for use in the present invention include any pharmaceutically acceptable sugar. The sugar may be a mono, di- or polysaccharide. In certain embodiments, the sugar may be, e.g., lactose, fructose, dextrose, sucrose, maltose, Candex (Emdex®, dextrates), dextrose/maltodextrin, and the like, and mixtures thereof. In a preferred embodiment of the invention, the sugar is fructose. The structural formula of fructose is provided below.

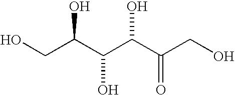

Fructose is a levorotatory monosaccharide and an isomer of glucose (C6H12O6). The chemical composition of fructose is (C₆H₁₂O₆). Pure fructose has a sweet taste similar to cane sugar, but with a “fruity” aroma. Fructose is the sweetest naturally occurring sugar, approximately 1.2× sweeter than sucrose. Fructose has a very low Glycemic Index (GI) relative to cane sugar (sucrose) and is metabolized by humans by a different metabolic pathway. Fructose is highly crystalline and poorly compressible. It also has a greater solubility than sucrose. Fructose is widely commercially available, e.g., Krystar® from Tate & Lyle, London, England; and from Spectrum Chemicals and Laboratory Products, New Brunswick, N.J.

### Polyol/Sugar Blend Component

In certain preferred embodiments of the invention, one or more polyols is blended with one or more sugars. This dry blend is then contacted with the atomized slurry in the drying environment to for the agglomerated excipient particles of the invention. Blending of the polyol(s) and sugar(s) may be accomplished with any suitable pharmaceutically acceptable mixer, e.g., a Turbula High Shear mixer. The blend is then contacted with the cellulose, silicon dioxide and disintegrant components to form the dry agglomerated excipient particles of the invention.

Alternatively, the polyol and sugar can be dry added to the atomized slurry separately without being blended.

In certain other embodiments, a portion of the polyol and sugar are blended together and a portion are added separately

In certain preferred embodiments, the polyol(s) and sugar(s) are first screened. Preferably, a 20 mesh screen is utilized prior to blending. The dry powder polyol and sugar are then e.g., fed into a drying chamber utilizing a feeder such as a Schenk AccuRate feeder and contacted with the atomized slurry in a drying chamber of a spray drying apparatus to obtain the agglomerated excipient particles of the invention.

The ratio of the polyol component to the sugar component is from about 99.1:0.9 to about 0.9:99.1. In other embodiments, the ratio of the polyol component to the sugar component is from about 80:20 to about 20:80. In certain preferred embodiments, the ratio of the polyol component to the sugar component is from about 60:40 to about 40:60. In certain more preferred embodiments, the ratio of the polyol component to the sugar component is from about 55:45% to about 45:55. In still other preferred embodiments, the ratio of the polyol component to the sugar component is about 1:1. In certain embodiments, the polyol/sugar dry blend and the blend prepared for tableting are prepared in a Turbula high shear mixer.

### Disintegrant Component

Any pharmaceutically acceptable disintegrant is contemplated for use in the present invention. Disintegrants suitable for use in the present invention may include, but are not limited to, starches, starch derivatives (e.g., low substituted carboxymethylcelulose starches, hydroxypropyl starch, etc.), clays (e.g., Veegum® HV and Bentonite®, etc.), celluloses (e.g., purified cellulose, methylcellulose, sodium carboxymethylcellulose, carboxymethylcellulose, microcrystalline cellulose, silicified microcrystalline cellulose, etc.), alginates (e.g., alginic acid, sodium alginate, etc.), pregelatinized corn starches, gums (e.g., agar, guar, karaya, tragacanth, etc.), surfactants, resins, effervescent mixtures, polyvinylpyrrolidone, cross-linked polyvinyl pyrrolidone, complex silicates, etc. The amount of disintegrant component may vary in a range from about 0.1% to about 99%, from about 0.5 to about 50%; from about 1 to about 25%; from about 2 to about 10%; from about 4 to about 6%, and from about 5% of the dry weight of the excipient of the present invention. A particularly preferred disintegrant is crospovidone XL.

### Active Agents

Certain embodiments of the invention further relate to ODT formulations which incorporate the ODT excipient of the present invention. In such embodiments, a pharmaceutically acceptable and/or nutritionally acceptable agent is contemplated by the present invention incorporated into the solid dosage form into a (e.g., therapeutically) effective amount as will be understood by one of ordinary skill in the art. In certain embodiments, the (e.g., active) agent is not adversely affected by the components of the solid dosage form. Exemplary active agents include: vitamins, minerals, plant derived components, flavinoids, proteins, amino acids, breath fresheners, vitamins and other dietary supplements, minerals, caffeine, nicotine, fruit juices, and the like, and mixtures thereof. Examples of useful drugs include ace-inhibitors, antianginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, biological response modifiers, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies such as sildenafil citrate, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids such as bromocryptine or nicotine, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, terine relaxants, anti-obesity drugs, erythropoietic drugs, anti-asthmatics, cough suppressants, mucolytics, DNA and genetic modifying drugs, and combinations thereof.

### Other Ingredients

Prior to being incorporated into a solid dosage form, the agglomerated particles may be combined with additional pharmaceutically acceptable excipients such as those described in the Handbook of Pharmaceutical Excipients, American Pharmaceutical Association, 4th Edition (2003), the disclosure of which is hereby incorporated by reference. Examples of suitable pharmaceutically acceptable excipients include, but are not limited to, binders, diluents, disintegrators, lubricants, preserving agents, fillers, surfactants and wetting agents, emulsifying agents, suspending agents, sweetening agents, flavoring agents, perfuming agents, and dispensing agents, etc.

In other embodiments, stabilizing agents are added to the solid dosage formulation.

### Binders

Binders suitable for use in the present invention include, but are not limited to, acacia, alginic acid, tragacanth, sucrose, gelatin, glucose, starch, cellulose derivatives (e.g., methyl cellulose, sodium carboxymethylcelulose), hydroxypropylmethylcellulose, ethyl cellulose, polyvinylpyrrolidone (PVP), sodium alginate, polyethyleneglycols, guar gum, polysaccharide acids, bentonites, the mixtures thereof, etc.

### Diluents

Diluents suitable for use in the present invention include, but are not limited to, pharmaceutically accepted hydrogels such as alginate, chitosan, methylmethacrylates, a monosaccharide, a disaccharide, a polyhydric alcohol, a cellulose or derivatives thereof (microcrystalline cellulose, hydroxypropyl cellulose, hydroxypropyl methyl cellulose, carboxymethylcellulose, ethylcellulose), agarose and Povidone™, kaolin, magnesium stearate, starch, lactose, sucrose, density-controlling agents such as barium sulfate and oils, dissolution enhancers such as aspartic acid, citric acid, glutamic acid, tartartic acid, sodium bicarbonate, sodium carbonate, sodium phosphate, glycine, tricine and TRIS. In certain embodiments the diluent may be an augmented microcrystalline cellulose as disclosed in U.S. Pat. No. 5,585,115, the disclosure of which is hereby incorporated by reference.

In certain embodiments, part or all of the diluent may comprise a pre-manufactured direct compression diluent. Suitable pre-manufactured direct compression diluents include, but are not limited to, Emcocel® (microcrystalline cellulose, N.F.), Emdex® (dextrates, N.F.), and Other direct compression diluents include anhydrous lactose (Lactose N.F., anhydrous direct tableting) from Sheffield Chemical, Union, N.J. 07083; Elcema® G-250 (Powdered cellulose, N.F.) from Degussa, D-600 Frankfurt (Main) Germany; Fast-Flo Lactose® (Lactose, N.F., spray dried) from Foremost Whey Products, Banaboo, Wis. 53913; Maltrin® (Agglomerated maltodextrin) from Grain Processing Corp., Muscatine, Iowa 52761; Neosorb 60® (Sorbitol, N.F., direct-compression) from Roquette Corp., 645 5th Ave., New York, N.Y. 10022; Nu-Tab® (Compressible sugar, N.F.) from Ingredient Technology, Inc., Pennsauken, N.J. 08110; Poly plasdone XL® (Crospovidone, N.F., cross-linked polyvinylpyrrolidone) from GAF Corp., New York, N.Y. 10020; Primojel® (Sodium starch glycolate, N.F., carboxymethyl starch) from Generichem Corp., Little Falls, N.J. 07424; Solka Floc® (Cellulose floc) from International Fiber Corp., N.Y., Spray-dried lactose® (Lactose N.F., spray dried) from Foremost Whey Products, Baraboo, Wis. 53913 and DMV Corp., Vehgel, Holland; and Sta-Rx 15000 (Starch 1500) (Pregelatinized starch, N.F., compressible) from Colorcon, Inc., West Point, Pa. 19486.

### Lubricants

Lubricants suitable for use in the present invention include, but are not limited to, a metallic stearate (e.g., magnesium stearate, calcium stearate, sodium stearate, etc.), stearic acid, talc, waxes, surfactants (e.g., sodium lauryl sulfate, magnesium lauryl sulfate, etc.), starch, silica, high molecular weight polyethylene glycols, etc. When the lubricant utilized is a metallic stearate, a metal concentration of the formulation/composition is more than 1 ppm. The lubricant may comprise, for example, magnesium stearate in any amount of about 0.5-3% by weight of the solid dosage form. In a particular preferred embodiment, the lubricant is sodium stearyl fumarate, (PRUV®, available from JRS Pharma LP).

### Surfactants

Surfactants or wetting agents suitable for use in the present invention include, but are not limited to, anionic surfactants, cationic surfactants, amphoteric (amphipathic/amphophilic) surfactants, and non-ionic surfactants. Examples of suitable surfactant or wetting agents include, inter alia, alkali metal chlorides, magnesium chloride, calcium chloride, organic acids such as citric, succinic, fumaric, malic, maleic, glutaric, lactic and the like, alkali metal sulfates such as sodium sulfate, alkali metal alkyl sulfates wherein the alkyl group is from 1 to 14 carbon atoms, such as sodium methyl sulfate, sodium lauryl sulfate and the like as well as dioctyl sodium sulfosuccinate, dihydrogen sodium phosphate, monohydrogen sodium phosphate, disodium hydrogen phosphate, sodium chloride, sodium fluoride and mixtures thereof, polyethyleneglycols as esters or ethers, polyethoxylated castor oil, polyethoxylated hydrogenated castor oil, polyethoxylated fatty acid from castor oil or polyethoxylated fatty acid from castor oil or polyethoxylated fatty acid from hydrogenated castor oil. Commercially available wetting agents which can be used are known under trade names Cremophor, Myrj, Polyoxyl 40 stearate, Emerest 2675, Lipal 395 and PEG 3350.

### Taste-Masking Agents and Taste-Masking Coatings

In certain embodiments, the active agent is coated sufficient to provide taste masking. Examples of a pharmaceutically acceptable film coatings include hydrophobic materials such as hydrophobic cellulosic materials including ethyl cellulose and hydrophilic materials such as hydrophillic cellulose materials such as hydroxypropylcellulose. In certain other embodiments, addition suitable taste-masking agents are added to the blend prior to tableting such as sodium bicarbonate, ion-exchange resins, cyclodextrin inclusion compounds, adsorbates, and the like.

### Modified Release Coating

In certain embodiments of the invention, the active agent is coated with a sufficient amount of a hydrophobic polymer to render the formulation capable of providing a release of the medicament such that a 12 or 24 hour formulation is obtained. In other embodiments of the present invention, the tablet or agglomerated excipient particle coating may comprise an enteric coating material in addition to or instead or the hydrophobic polymer coating. Examples of suitable enteric polymers include cellulose acetate phthalate, hydroxypropylmethylcellulose phthalate, polyvinylacetate phthalate, methacrylic acid copolymer, shellac, hydroxypropylmethylcellulose succinate, cellulose acetate trimellitate, and mixtures of any of the foregoing. An example of a suitable commercially available enteric material is available under the trade name Eudragit™ L 100-555.

### Film Coating

Film-coated tablets are easier to swallow than uncoated tablet cores, are usually easier to distinguish from other tablets—in particular when the film-coat contains a dye or a pigment—and may furthermore have an improved stability (shelf-life). In the instant case, a mixture comprising a film-forming polymer and a plasticizer, for example, hydroxypropyl methylcellulose with or without a polyethylene glycol, e.g. macrogol 6000, may be employed for film-coating tablet cores. Of particular importance in the case of fast-dissolving tablets, is the requirement that the film-coat should not adversely affect the disintegration and dissolution of the active ingredient from the tablet. Therefore, the weight of the film-coat conveniently is in the range of e.g., 0.05% to 8% of the uncoated tablet core. For example, a useful coating polymer is hydroxypropyl methylcellulose applied from an aqueous solution in an amount of about 1.5% to 5% based on the weight of the tablet core.

In further embodiments, the active agent may be coated with a hydrophilic coating in addition to or instead of the above-mentioned coatings. An example of a suitable material which may be used for such a hydrophilic coating is hydroxypropylmethylcellulose (e.g., Opadry®, commercially available from Colorcon, West Point, Pa.).

### Coating Processes

Coatings described herein may be applied in any pharmaceutically acceptable manner known to those skilled in the art. For example, in one embodiment, the coating is applied via a fluidized bed or in a coating pan. In one embodiment, the coated active agent particle may be dried, e.g., at about 60°-70° C. for about 3-4 hours in a coating pan. The solvent for the hydrophobic polymer or enteric coating may be organic, aqueous, or a mixture of an organic and an aqueous solvent. The organic solvents may be, e.g., isopropyl alcohol, ethanol, and the like, with or without water.

The coatings which may be optionally applied to the active agent may comprise from about 0.5% to about 30% by weight of the final solid dosage form.

In certain embodiments of the present invention, an additional dose of the active agent may be included in either the hydrophobic or enteric coating, or in an additional overcoating coated on the outer surface of the active agent core (without the hydrophobic or enteric coating) or as a second coating layer coated on the surface of the base coating comprising the hydrophobic or enteric coating material. This may be desired when, for example, a loading dose of a therapeutically active agent is needed to provide therapeutically effective blood levels of the active agent when the formulation is first exposed to gastric fluid. The loading dose of active agent included in the coating layer may be, e.g., from about 10% to about 40% of the total amount of medicament included in the formulation.

### Coloring Agents

The solid dosage forms of the present invention may also contain effective amounts of coloring agents, (e.g., titanium dioxide, F.D. & C. and D. & C. dyes; see the Kirk-Othmer Encyclopedia of Chemical Technology, Vol. 5, pp. 851-884, hereby incorporated by reference), stabilizers, binders, odor controlling agents, and preservatives.

### Flavoring Agents

The solid dosage forms of the present invention may also comprise one or more pharmaceutically acceptable flavoring agents. A non-limiting list includes: mint, raspberry, licorice, orange, lemon, grapefruit, caramel, vanilla, cherry, grape flavors, tutti frutti, combinations thereof, and the like.

### pH Modifiers

Suitable pH modifiers for use in the present invention include citric acid, tartaric acid, phosphoric acid, hydrochloric acid, maleic acid, sodium hydroxide, and the like.

### Sweeteners

Suitable sweeteners include aspartame, acesulfame potassium, sucralose, saccharin, saccharin sodium, xylitol, thaumatic, combinations thereof, and the like.

It is recognized that pharmaceutical excipients may perform more than one function, and are therefore characterized as having different uses depending on the particular application. While the use of an excipient in the context of a particular formulation may determine the function of the excipient, the inclusion of any particular excipient into any one or more category as set forth above is not meant to limit the function of that excipient.

### Tableting

As previously mentioned, the ODT excipient of the present invention may in certain preferred embodiments be combined with one or more active agents, (e.g. therapeutic agents, nutraceutical agent) and other optional pharmaceutically acceptable excipients and coprocessed into (ODT) tablets. The aforementioned mixture, in an amount sufficient to make a uniform batch of tablets, may then be subjected to tableting. Tableting force should be sufficient to create tablets having suitable hardness and low friability, e.g., less than 2%, while also allowing for disintegration for ODT solid dosage forms as described herein. Preferably, the solid dosage forms have a hardness from about 2 to about 9 kP, preferably about 3 to about 8 KP and more preferably about 4 kP.

### Tablet Size

The average tablet size for round tablets is preferably about 50 mg to 1000 mg. In certain preferred embodiments, the tablets are about 500 mg or less. Other formulations prepared in accordance with the present invention may be suitably shaped for other uses or locations, such as other body cavities, e.g., periodontal pockets, surgical wounds, vaginally. It is contemplated that for certain uses, e.g., antacid tablets, vaginal tablets and possibly implants, that the tablet will be larger.

### Equipment for Preparing Agglomerated Excipient Particles

Any device capable of producing atomized particles and a sufficient volume of warm air is contemplated for use in preparing the excipients according to the present invention. In a preferred embodiment, the excipients are prepared in a spray dryer and the dry powder polyol/sugar blend is introduced with a feeder. In certain embodiments, a Niro Production Minor Spray Dryer is utilized. In certain preferred embodiments, a commercial scale Spray Dryer is utilized. In certain other embodiments, a Schenck feeder is utilized for adding the dry powder blend of polyol/sugar into the drying chamber.

Any suitable apparatus for compressing the blend comprising the agglomerated excipient particles, active agent and optional additional excipients are contemplated by the present invention. In a particular preferred embodiment, a Riva Piccola tablet press with a gravity feeder attachment is used to form the solid dosage forms. In still other embodiments, ⅝″ lozenge shape tooling is used. In yet further preferred embodiments, 7/16″ round deep concave tooling is used. In still other embodiments, 7/16″ round deep concave tooling is used.

### ADVANTAGES OF THE PRESENT INVENTION

It is desired that the agglomerated excipient particles in accordance with certain embodiments of the present invention described above provide a number of advantages. Specifically, the agglomerated particles are desired to provide superior flow characteristics to prior art compositions. As one of ordinary skill in the art will appreciate, superior flow characteristics allow faster and more efficient processing for tablets, capsules, and other dosage forms.

In another aspect of the invention, it is also desired that the agglomerated excipient particles in accordance with certain embodiments of the present invention provide superior compaction characteristics to prior art compositions. As one of ordinary skill in the art will appreciate, the superior compaction characteristics allow faster and more efficient processing for tablets, and, moreover, allow a larger percentage of an active agent component to be included in each tablet.

In other aspects of the invention, it is desired that the agglomerated excipient particles in accordance with certain embodiments of the present invention exhibit superior content uniformity when tableted than to prior art compositions.

As set forth in certain the examples describing particular embodiments of the invention, another advantage of the agglomerated excipient particles of the present invention involves lower compression forces needed to create solid dosage forms, i.e., tablets that have sufficient hardness and acceptable low friability, e.g., 2%, while still exhibiting sufficient rapid disintegration when placed on the tongue or when tested according to USP disintegration testing methods.

### DETAILED DESCRIPTION OF PREFERRED EMBODIMENTS

The following examples illustrate various aspects of the present invention, and are set forth to assist in understanding the invention. These examples should not be construed as specifically limiting the invention described and claimed herein. Variations of the invention, including the substitution of all equivalents now known or later developed, which would be within the purview of those skilled in the art, and changes in formulation or minor changes in experimental design, are considered to fall within the scope of the invention and appended claims.

### Preparation of Excipients

Exemplary materials used in preparing certain excipients have been sourced from suppliers as designated in Table 2.

TABLE 2
Sourced materials to be used in ODT excipient product

| Material | Tradename | Source |
|---|---|---|
| Mannitol | Pearlitol ® 50C | Mutchler |
| Sorbitol | Neosorb ® P60W | Mutchler |
| Xylitol | Xylisorb ® 300 | Mutchler |
| Dextrose | Candex ® | JRS Pharma |
| Silicified Microcrystalline | e.g., Prosolv ® HD 50 | JRS Pharma |
| Cellulose | | |
| Rayonier pulp-sulfatate slurry | n/a | JRS Pharma |
| Colloidal Silicon Dioxide | Cabosil ® M-5-P | Cabot |

### Example 1

In Example 1, a dry addition procedure to determine dry powder feed rate for fructose was performed. Fructose has a favorable sweetness profile, having 50% more sweetness than sucrose. Utilizing a Niro pump, stop watch, Schenck dry addition feeder and balance, the pump rate of water was determined by adjusting the pump rate to various levels, collecting material passed through the pump and weighing the material. The weights of materials produced at various pump rate levels were recorded. Utilizing the same method, the pump rate with the MCC slurry containing about 15% solids was determined. Each trial lasted 2 minutes.

The results for 5 feed rate trials for dry powder fructose are set forth below in Table 3.

TABLE 3

| | | | | g/m |
|---|---|---|---|---|
| | | | | adjusted |
| | | Trial Time | | for 0.5% |
| Feed Rate | Grams | min | g/m | water |
| 125 | 8.5 | 2 | 4.25 | 4.23 |
| 175 | 64 | 2 | 32.4 | 32.24 |
| 200 | 96 | 2 | 48 | 47.76 |
| 225 | 121.2 | 2 | 60.6 | 60.30 |
| 250 | 150.3 | 2 | 75.15 | 74.77 |

Using the equation from the linear trend line (y=0.5643x−68.174 R²=0.9995), the values in table 4 were obtained.

TABLE 4
Fructose Dry Powder Feed Rate Adjusted for Moisture Content of 50%

| G/min fructose | Feed rate |
|---|---|
| 5 | 126 |
| 6 | 128 |
| 7 | 130 |
| 8 | 131 |
| 9 | 133 |
| 10 | 135 |
| 11 | 137 |
| 12 | 139 |
| 13 | 140 |
| 14 | 142 |
| 15 | 144 |
| 16 | 146 |
| 17 | 147 |
| 18 | 149 |
| 19 | 151 |
| 20 | 153 |
| 21 | 154 |
| 22 | 156 |
| 23 | 158 |
| 24 | 160 |
| 25 | 162 |
| 26 | 163 |
| 27 | 165 |
| 28 | 167 |
| 29 | 169 |
| 30 | 170 |
| 31 | 172 |
| 32 | 174 |
| 33 | 176 |
| 34 | 178 |
| 35 | 179 |
| 36 | 181 |
| 37 | 183 |
| 38 | 185 |
| 39 | 186 |
| 40 | 188 |
| 41 | 190 |
| 42 | 192 |
| 43 | 193 |
| 44 | 195 |
| 45 | 197 |
| 46 | 199 |
| 47 | 201 |
| 48 | 202 |
| 49 | 204 |
| 50 | 206 |
| 51 | 208 |
| 52 | 209 |
| 53 | 211 |
| 54 | 213 |
| 55 | 215 |
| 56 | 217 |
| 57 | 218 |
| 58 | 220 |
| 59 | 222 |
| 60 | 224 |
| 61 | 225 |
| 62 | 227 |
| 63 | 229 |
| 64 | 231 |
| 65 | 232 |
| 66 | 234 |
| 67 | 236 |
| 68 | 238 |
| 69 | 240 |
| 70 | 241 |
| 71 | 243 |
| 72 | 245 |
| 73 | 247 |
| 74 | 248 |
| 75 | 250 |
| 76 | 252 |
| 77 | 254 |
| 78 | 255 |
| 79 | 257 |
| 80 | 259 |
| 81 | 261 |
| 82 | 263 |
| 83 | 264 |
| 84 | 266 |
| 85 | 268 |
| 86 | 270 |
| 87 | 271 |
| 88 | 273 |
| 89 | 275 |
| 90 | 277 |
| 91 | 279 |
| 92 | 280 |
| 93 | 282 |
| 94 | 284 |
| 95 | 286 |
| 96 | 287 |
| 97 | 289 |
| 98 | 291 |
| 99 | 293 |
| 100 | 294 |

This chart is used to match a desired ratio of fructose to cellulose delivered by the feed pump. This chart is necessary for in process changes resultant of drier temperature changes.

### Example 2

In Example 2, MCC, CSD and sodium starch glycolate were spray dried along with dry addition fructose sourced from Spectrum as set forth in Table 5.

TABLE 5

| | | Batch | Slurry solid |
|---|---|---|---|
| Ingredient | % of formulation | size (kg) l | contribution |
| MCC | 46.5 | 0.465 | 10.86 |
| CSD | 2 | 0.02 | 0.47 |
| Explotab | 5 | 0.05 | 1.17 |
| Total | 53.5 | 0.535 | 12.5 |
| Dry added fructose | 46.5 | 0.465 | |
| Powder Total | 100 | 1.0 | |
| Solids content of MCC | 18.33% | | |
| slurry - sulfatate | | | |
| Required weight of MCC | 2.54 | | |
| slurry (kg) | | | |
| MCC solids target | 10.86% | | |
| Required water added (kg) | 1.74 | | |
| Total water weight (kg) | 3.82 | | |
| Total slurry weight (kg) | 4.28 | | |
| Overall slurry solids target | 12.5% | | |

The batch was prepared by adding sodium starch glycolate to MCC slurry in fractions. CSD was slowly added to the slurry mixture, and water added in fractions as necessary to make a workable slurry. Finally, the slurry was spray dried at an inlet temperature of 200 degrees temp and outlet temperature of 100 degrees at 55 Hz. The damper was set to the one position from full open. A small (2.5″) dry addition gap was used. The particle target size was 65 μM.

This run was successful in terms of yield. Mouth feel and sweetness were also determined to be successful.

### Example 3

In Example 3, a fructose/mannitol 1:1 mixture feed rate was determined according to the process set forth in Example 1. Results are set forth in Table 6.

TABLE 6

| | | | | | | g/m |
|---|---|---|---|---|---|---|
| | | Trial time | | Agitator | | adjusted for |
| Feed rate | Grams | min. | Agitator | rate | g/m | 0.5% water |
| 150 | 30 | 2 | Y | 350 | 15 | 14.93 |
| 200 | 70.8 | 2 | Y | 350 | 35.4 | 35.22 |
| 250 | 112.7 | 2 | Y | 350 | 56.35 | 56.07 |
| 275 | 133.6 | 2 | Y | 350 | 66.8 | 66.47 |
| 300 | 156.3 | 2 | Y | 350 | 78.15 | 77.76 |

As in Example 1, utilizing the formula y=0.4174x−48.005 R²=0.9997, dry powder feed rate of fructose/mannitol 1:1 adjusted for moisture content 0.5% values were obtained.

### Example 4

In Example 4, an Emdex/mannitol 1:1 mixture feed rate was determine according to the process set forth in Example 1. Results are set forth in Table 7.

TABLE 7

| | | Trial | | | | g/m |
|---|---|---|---|---|---|---|
| | | time | | Agitator | | adjusted for |
| Feed rate | Grams | min. | Agitator | rate | g/m | 0.5% water |
| 150 | 28 | 2 | Y | 350 | 14 | 13.34 |
| 200 | 65.4 | 2 | Y | 350 | 3532.7.4 | 31.15 |
| 250 | 100.4 | 2 | Y | 350 | 56.3550.2 | 47.82 |
| 275 | 121.5 | 2 | Y | 350 | 60.75 | 57.86 |
| 300 | 156.6 | 2 | Y | 350 | 78.3 | 74.58 |

As in Example 1, utilizing the formula y=0.3505x−39.173 R²=0.9996, dry powder feed rate of Emdex/mannitol 1:1 adjusted for moisture content 4.75% values were obtained.

This mixture was then dry added to a slurry of MCC, CSD and Explotab as set forth in Table 8.

TABLE 8

| | | Batch | Slurry solid |
|---|---|---|---|
| Ingredient | % of formulation | size (kg) l | contribution |
| MCC | 46.5 | 0.465 | 10.43 |
| CSD | 2 | 0.02 | 0.45 |
| Explotab | 5 | 0.05 | 1.12 |
| Total | 53.5 | 0.535 | 12 |
| Dry added | 46.5 | 0.465 | |
| Emdex/mannitol | | | |
| Powder Total | 100 | 1.0 | |
| Solids content of MCC | 18.33% | | |
| slurry - sulfatate | | | |
| Required weight of MCC | 2.54 | | |
| slurry (kg) | | | |
| MCC solids target | 10.43% | | |
| Required water added (kg) | 1.92 | | |
| Total water weight (kg) | 3.99 | | |
| Total slurry weight (kg) | 4.48 | | |
| Overall slurry solids target | 12% | | |

The batch was prepared by adding CSD to MCC slurry and adding required water. Explotab was slowly added to MCC/CSD. Water was added in fractions if necessary to make a workable slurry. Finally, spray drying at temperatures of 200 degrees inlet and 100 degrees outlet at 55 Hz was performed. The damper was set to “one” position from full open. A small (2.5″) dry addition gap was used. The particle target size was 65 μM. This run was successful and the material was bagged.

### Example 5

In Example 5, the material obtained from Examples 3 and 4 was each used to create tablets. Each powder was lubricated with 0.5% PRUV® (20 mesh screened) and were blended in a Turbula high shear mixer for 5 minutes.

Compaction involved a 0.5 inch flat faced, 0.5 inch standard concave and 0.5 inch lozenge tableting tools. A Piccola press was set at 25 rpm with powder feeder set to 5. The average tablet target weight was 600 mg. Tablet hardness was tested using a ERWEKA TBH-30 and disintegration was tested utilizing a ERWEKA CT-62. The results are set forth in Table 9 below.

TABLE 9

| | | | 10 tab | | | | | | | | | Average |
|---|---|---|---|---|---|---|---|---|---|---|---|---|
| | Average | Std | weight | Average | Std | Disintegration | | | | | | Disintegration |
| Tablet | Kp | Deviation | (mg) | kN | Deviation | Time (sec) | | | | | | Time (sec) |
| 1 | 2.69 | 0.24 | 5959.4 | 10.86 | 1.065 | 19 | 21 | 23 | 21 | 25 | 21 | 22 |
| 2 | 2.61 | 0.11 | 5957.4 | 9.527 | 0.26 | 17 | 23 | 17 | 19 | 23 | 11 | 18 |
| 3 | 2.28 | 0.09 | 5954.7 | 8.062 | 0.104 | 11 | 15 | 15 | 13 | 19 | 9 | 14 |
| 4 | 2.51 | 0.27 | 6056.7 | 3.991 | 0.119 | 15 | 19 | 19 | 19 | 21 | 15 | 18 |
| 5 | 2.69 | 0.12 | 5957 | 4.101 | 0.089 | 23 | 23 | 23 | 17 | 27 | 19 | 22 |
| 6 | 2.14 | 0.12 | 5999.8 | 3.449 | 0.092 | 19 | 21 | 13 | 11 | 23 | 15 | 17 |

All tablets disintegrated in under 30 seconds regardless of tooling or formulation. It took at least twice the compaction force to create 2-3 kp tablets with fructose (Example 3) than Emdex (Example 4).

### Example 6

In Example 6, ibuprofen formulations utilizing the material from Example 4 were prepared according to Table 10. The tablets also contained a sodium stearyl fumarate lubricant, (PRUV®, available from JRS Pharma LP)

TABLE 10

| | | | | |
|---|---|---|---|---|
| | | | | Blend |
| | | Tablet | | Required |
| Component | Formulation | (mg) | Percent | Amount (g) |

Formulation A 1:1 ODT:Drug

| | | | | | | |
|---|---|---|---|---|---|---|
| ODT | Example 4 | Fructose | 80% | 250.175 | 43.89 | 43.89 |
| Drug | Balchem 80% | 200 mg | | 250 | 43.86 | 43.86 |
| | Ibuprofen | dose | | | | |
| Lubricant | Pruv | | | 11.4 | 2 | 2 |
| Anti-adherent | Syloid | | | 28.5 | 5 | 5 |
| Disintegrant | Crospovidone XL | | | 28.5 | 5 | 5 |
| flavor | Tutti-frutti | | | 1.425 | 0.25 | 0.25 |
| | | | Target total | 570 | 100 | 100 |
| | | | Total | 570 | 100 | 100 |

Formulation B 1.5:1 ODT:Drug

| | | | | | | |
|---|---|---|---|---|---|---|
| ODT | Example 4 | Fructose | 80% | 381.8 | 53.03 | 53.03 |
| Drug | Balchem 80% | 200 mg | | 250 | 34.72 | 34.72 |
| | Ibuprofen | dose | | | | |
| Lubricant | Pruv | | | 11.414 | 2 | 2 |
| Anti-adherent | Syloid | | | 28.536 | 5 | 5 |
| Disintegrant | Crospovidone XL | | | 28.536 | 5 | 5 |
| flavor | Tutti-fruitti | | | 1.425 | 0.25 | 0.25 |
| | | | Target total | 720 | 100 | 100 |
| | | | Total | 720 | 100 | 100 |

Formulation C 2:1 ODT:Drug

| | | | | | | |
|---|---|---|---|---|---|---|
| ODT | Example 4 | Fructose | 80% | 495.875 | 58.34 | 58.34 |
| Drug | Balchem 80% | 200 mg | | 250 | 29.41 | 29.41 |
| | Ibuprofen | dose | | | | |
| Lubricant | Pruv | | | 17 | 2 | 2 |
| Anti-adherent | Syloid | | | 42.5 | 5 | 5 |
| Disintegrant | Crospovidone XL | | | 42.5 | 5 | 5 |
| flavor | Tutti-fruitti | | | 2.125 | 0.25 | 0.25 |
| | | | Target total | 850 | 100 | 100 |
| | | | Total | 850 | 100 | 100 |

Tablets were prepared utilizing a Riva Piccola gravity feed press at 25 rpm with ⅝ inch tooling, round, flat faced tooling. An ERWEKA TBH-30 tablet hardness tester and an ERWEKA CT-62 tablet disintegrator were also used. Hardness for formulations A-C is set forth in Table 11 below.

TABLE 11

| | | | |
|---|---|---|---|
| | Hardness | Thickness | Diameter |
| Tablet # | (kP) | (mm) | (mm) |

Formulation A
Compression force 10.999

| | | | |
|---|---|---|---|
| 1 | 2.34 | 2.52 | 15.86 |
| 2 | 2.55 | 2.51 | 15.87 |
| 3 | 2.96 | 2.51 | 15.87 |
| 4 | 2.45 | 2.5 | 15.89 |
| 5 | 2.34 | 2.61 | 15.57 |
| 6 | 2.55 | 2.55 | 15.88 |
| 7 | 2.55 | 2.53 | 15.88 |
| 8 | 2.55 | 2.52 | 15.87 |
| 9 | 2.24 | 2.51 | 15.85 |
| 10 | 2.24 | 2.52 | 15.87 |
| Average | 2.48 | 2.53 | 15.84 |
| Std. | 0.21 | 0.03 | 0.10 |
| Deviation | | | |

Formulation B
Compression force 9.809

| | | | |
|---|---|---|---|
| 1 | 2.04 | 3.21 | 15.89 |
| 2 | 2.14 | 3.21 | 15.87 |
| 3 | 2.65 | 3.21 | 15.88 |
| 4 | 2.14 | 3.23 | 15.9 |
| 5 | 2.04 | 3.23 | 15.89 |
| 6 | 2.55 | 3.22 | 15.89 |
| 7 | 2.04 | 3.21 | 15.88 |
| 8 | 2.04 | 3.22 | 15.89 |
| 9 | 2.24 | 3.21 | 15.89 |
| 10 | 2.24 | 3.2 | 15.89 |
| Average | 2.21 | 3.22 | 15.89 |
| Std. | 0.22 | 0.01 | 0.01 |
| Deviation | | | |

Formulation C
Compression force 9.462

| | | | |
|---|---|---|---|
| 1 | 2.45 | 3.85 | 15.91 |
| 2 | 2.04 | 3.9 | 15.92 |
| 3 | 2.34 | 3.85 | 15.9 |
| 4 | 2.24 | 3.88 | 15.91 |
| 5 | 2.15 | 3.87 | 15.9 |
| 6 | 2.14 | 3.86 | 15.91 |
| 7 | 2.45 | 3.87 | 15.91 |
| 8 | 2.34 | 3.86 | 15.91 |
| 9 | 2.34 | 3.88 | 15.9 |
| 10 | 2.14 | 3.88 | 15.91 |
| Average | 2.26 | 3.87 | 15.91 |
| Std. | 0.14 | 0.02 | 0.01 |
| Deviation | | | |

After compaction, tablets were tested for disintegration at 44% relative humidity and 20.8° C. ambient temperature. The results are set forth in Table 12 below.

TABLE 12

| | Type | | | | | | | | | | | | |
|---|---|---|---|---|---|---|---|---|---|---|---|---|---|
| | ⅝″ | | | 10 tab | | | | | | | | | Average |
| | flat | Average | Std | wt | Average | Std | Disintegration time | | | | | | Disintegration |
| Sample | faced | Kp | Deviation | (mg) | kN | Deviation | (sec) | | | | | | time (sec) |
| A | FF | 2.477 | 0.21 | 5585.4 | 10.999 | 0.284 | 21 | 35 | 25 | 45 | 43 | 33 | 34 |
| B | FF | 2.212 | 0.22 | 7098.3 | 9.809 | 0.259 | 23 | 27 | 17 | 31 | 31 | 33 | 27 |
| C | FF | 2.263 | 0.14 | 8541.4 | 9.462 | 0.277 | 15 | 31 | 17 | 33 | 35 | 19 | 25 |

Formulation C (2:1) ODT:drug required the least amount of force to create a tablet of desired hardness, 2-3 kp and it also disintegrated the fastest.

### Example 7

In Example 7, the coprocessed formulation of Example 3 and a dry blend of the same constituents were compared for compaction, disintegration and sweetness to determine advantages of coprocessing. For blend 1, the material from Example 3 was blended with 0.25% PRUV in a high shear mixer for 5 minutes and transferred to a plastic bag.

Blend 2 contained:

| | |
|---|---|
| Prosolv HD50 | 27% |
| Fructose | 34% |
| Mannitol | 34% |
| Explotab | 5% |

The dry blend was prepared by first blending the fructose and mannitol for 5 minutes, then adding Prosolv HD50 and Explotab, and blending for another 5 minutes. Finally PRUV in an amount of 0.25% was added and blended for 5 minutes and transferred to a plastic bag.

Tablets were prepared and tested for hardness and disintegration as set forth in Example 7 after the desired hardness (2-3 kp) was achieved. Target weight was 500 mg. The results are provided in Table 13 below.

TABLE 13

| | | | |
|---|---|---|---|
| | Hardness | Thickness | Diameter |
| Tablet # | (kP) | (mm) | (mm) |

Blend 1 Coprocessed from Example 14 and PRUV
Compression force 6.578

| | | | |
|---|---|---|---|
| 1 | 2.14 | 3.86 | 12.63 |
| 2 | 1.94 | 3.85 | 12.63 |
| 3 | 2.04 | 3.87 | 12.62 |
| 4 | 1.53 | 3.88 | 12.64 |
| 5 | 2.14 | 3.8 | 12.63 |
| 6 | 2.04 | 3.86 | 12.63 |
| 7 | 1.83 | 3.88 | 12.62 |
| 8 | 2.04 | 3.87 | 12.63 |
| 9 | 2.14 | 3.87 | 12.63 |
| 10 | 2.04 | 3.89 | 12.63 |
| Average | 1.99 | 3.87 | 12.63 |
| Std. | 0.19 | 0.01 | 0.01 |
| Deviation | | | |

Blend 2 Dry Blend
Compression force 7.740

| | | | |
|---|---|---|---|
| 1 | 1.53 | 4.02 | 12.62 |
| 2 | 1.94 | 4.03 | 12.62 |
| 3 | 1.94 | 4.03 | 12.61 |
| 4 | 2.04 | 4.02 | 12.62 |
| 5 | 2.04 | 4.03 | 12.63 |
| 6 | 1.94 | 4.04 | 12.62 |
| 7 | 1.94 | 4.02 | 12.63 |
| 8 | 1.94 | 4.02 | 12.62 |
| 9 | 1.53 | 4.04 | 8.43 |
| 10 | 1.83 | 4.02 | 12.62 |
| Average | 1.90 | 4.03 | 12.62 |
| Std. | 0.15 | 0.01 | 0.01 |
| Deviation | | | |

After compaction, tablets were tested for disintegration at 45% relative humidity and 21.4° C. ambient temperature. The results are set forth in Table 14 below.

TABLE 14

| | Type | | | | | | | | | | | | Average |
|---|---|---|---|---|---|---|---|---|---|---|---|---|---|
| | ½″ | | | 10 tab | | | | | | | | | DisinTegration |
| | lozenge | Average | Std | wt | Average | Std | Disintegration time | | | | | | time |
| Sample | (L) | Kp | Deviation | (mg) | kN | Deviation | (sec) | | | | | | (sec) |
| Blend 1 | L | 1.99 | 0.19 | 5070.4 | 6.578 | 0.197 | 5 | 9 | 7 | 8 | 13 | 7 | 8 |
| Coprocessed | | | | | | | | | | | | | |
| Blend 2 | L | 1.90 | 0.15 | 4896.6 | 7.74 | 0.46 | 15 | 7 | 5 | 13 | 11 | 11 | 10 |
| Dry blend | | | | | | | | | | | | | |

Based on these results, the coprocessed material of Example 3 appears to have better compaction properties because it took less force to make harder tablets. The tablets prepared from the coprocessed material also disintegrated faster and had a sweeter taste.

### Example 8

In Example 8, a matrix containing a 1:1 ratio of fructose:mannitol, Explotab CLV, MCC and CSD was prepared having the formula set forth in Table 15.

TABLE 15

| | % of | Batch size | Slurry solid |
|---|---|---|---|
| Ingredient | formulation | (kg) 1.5 | contribution % |
| MCC | 46.5 | 0.6975 | 10.26 |
| CSD | 2 | 0.03 | 0.44 |
| Explotab CLV | 5 | 0.075 | 1.10 |
| Total | 53.5 | 0.8025 | 11.80 |
| Dry added | 46.5 | 0.6975 | |
| fructose:mannitol 1:1 | | | |
| Powder Total | 100 | 1.50 | |
| Solids content of MCC | 18.33% | | |
| slurry - sulfatate | | | |
| Required weight of MCC | 3.81 | | |
| slurry (kg) | | | |
| MCC solids target | 10.26% | | |
| Required water added (kg) | 3.00 | | |
| Total water weight (kg) | 6.10 | | |
| Total slurry weight (kg) | 6.80 | | |
| Overall slurry solids target | 11.80% | | |

The matrix was prepared by first adding CSD to the MCC slurry, and adding the required amount of water, then the Explotab was slowly added to the slurry, water is then added in fractions if necessary to make a workable slurry. The mixture is spray-dried at 200 inlet and 100 outlet at 55 Hz with the polyol mixture dry added. The damper was set to one up from full open position. A small 2.5″ dry addition gap was used. The pump was ran at 10, and the feeder was set to 189 (adjusted to 0.5% moisture content), with the agitator set to 350. Particle size target was 65 μM. The run was successful and the material was bagged.

### Example 9

In Example 9, the process of Example 8 was repeated, however the percentage of the fructose:mannitol 1:1 was increased to 70% as set forth in Table 16.

TABLE 16

| Ingredient | % of formulation |
|---|---|
| MCC | 23 |
| CSD | 2 |
| Explotab CLV | 5 |
| Total | 30 |
| Dry added | 70 |
| fructose:mannitol 1:1 | |
| Powder Total | 100 |
| Solids content of MCC | 17.6 |
| slurry - sulfatate | |
| Required weight of MCC | 1.31 |
| slurry (kg) | |
| MCC solids target | 9.2 |
| Required water added (kg) | 1.19 |
| Total water weight (kg) | 2.27 |
| Total slurry weight (kg) | 2.5 |
| Overall slurry solids target | 12 |

The mixture is spray-dried at 200 degrees Celsius inlet and 100 degrees Celsius outlet at 55 Hz with the polyol mixture dry added. The damper was set to one up from full open position. A small 2.5″ dry addition gap was used. The pump was ran at 10, and the feeder was set to 316 (adjusted to 0.5% moisture content), with the agitator set to 350. Particle size target was 65 μM. The run was successful and the material was bagged.

### Example 10

In Example 10, the process of Example 9 was repeated, however Explotab was replaced with crospovidone XL as set forth in Table 17.

TABLE 17

| Ingredient | % of formulation |
|---|---|
| MCC | 23 |
| CSD | 2 |
| Crospovidone XL | 5 |
| Total | 30 |
| Dry added | 70 |
| fructose:mannitol 1:1 | |
| Powder Total | 100 |
| Solids content of MCC | 17.5 |
| slurry - sulfatate | |
| Required weight of MCC | 1.34 |
| slurry (kg) | |
| MCC solids target | 9.2 |
| Required water added (kg) | 1.73 |
| Total water weight (kg) | 3.41 |
| Total slurry weight (kg) | 3.75 |
| Overall slurry solids target | 12 |

Mixing crospovidone XL into the mixture was easier and resulted in a lower viscosity slurry. The mixture is spray-dried at 200 degrees Celsius inlet and 100 degrees Celsius outlet at 55 Hz with the polyol mixture dry added. The damper was set to one up from full open position. A small 2.5″ dry addition gap was used. Particle size target was 65 μM. The run was successful. The dryer had minimal buildup and processability improved. The material was bagged.

### Example 11

In Example 11, the materials from Examples 9 and 10 were each blended with 0.5% PRUV which has been screened through a 20 mesh screen. The blends were each mixed in a Turbula high shear mixer for 5 minutes, and subsequently compressed into tablets. The results are set forth in Table 18 below.

TABLE 18

| | | | |
|---|---|---|---|
| | Hardness | Thickness | Diameter |
| Tablet # | (kP) | (mm) | (mm) |

Example 9 with 0.5% PRUV
Compression force 9.650

| | | | |
|---|---|---|---|
| 1 | 2.65 | 3.83 | 12.68 |
| 2 | 2.14 | 3.83 | 12.65 |
| 3 | 2.45 | 3.85 | 12.66 |
| 4 | 2.65 | 3.82 | 12.66 |
| 5 | 2.55 | 3.84 | 12.67 |
| 6 | 2.55 | 3.84 | 12.65 |
| 7 | 2.55 | 3.83 | 12.66 |
| 8 | 2.55 | 3.84 | 12.65 |
| 9 | 2.34 | 3.83 | 12.65 |
| 10 | 2.65 | 3.84 | 12.66 |
| Average | 2.51 | 3.84 | 12.66 |
| Std. | 0.16 | 0.01 | 0.01 |
| Deviation | | | |

Example 10 with 0.5% PRUV
Compression force 7.977

| | | | |
|---|---|---|---|
| 1 | 2.45 | 4.05 | 12.65 |
| 2 | 2.34 | 4.05 | 12.65 |
| 3 | 2.75 | 4.03 | 12.65 |
| 4 | 2.45 | 4.06 | 12.65 |
| 5 | 2.55 | 4.04 | 12.64 |
| 6 | 2.34 | 4.03 | 12.66 |
| 7 | 2.65 | 4.04 | 12.65 |
| 8 | 2.34 | 4.05 | 12.66 |
| 9 | 2.55 | 4.05 | 12.66 |
| 10 | 2.55 | 4.03 | 12.65 |
| Average | 2.5 | 4.04 | 12.65 |
| Std. | 0.14 | 0.01 | 0.01 |
| Deviation | | | |

After compaction, tablets were tested for disintegration at 35% relative humidity and 21.7° C. ambient temperature. The results are set forth in Table 19 below.

TABLE 19

| | Type | | | | | | | | | | | | |
|---|---|---|---|---|---|---|---|---|---|---|---|---|---|
| | ½″ | | | 10 tab | | | | | | | | | Average |
| | lozenge | Average | Std. | wt | Average | Std | Disintegration time | | | | | | Disintegration |
| Sample | (L) | Kp | Deviation | (mg) | kN | Deviation | (sec) | | | | | | time (sec) |
| Example | L | 2.51 | 0.16 | 4959.4 | 9.650 | 0.249 | 11 | 17 | 7 | 13 | 13 | 13 | 12 |
| 19 blend | | | | | | | | | | | | | |
| Example | L | 2.5 | 0.14 | 4971.6 | 7.977 | 0.158 | 11 | 17 | 3 | 5 | 9 | 7 | 9 |
| 20 blend | | | | | | | | | | | | | |

Seemingly, the blend using the material from Example 10, containing crospovidone XL used less compaction force to make a tablet of similar hardness which also disintegrated faster than tablets made utilizing Explotab.

### Example 12

In Example 12, the process of Example 10 was repeated, however Explotab was replaced with Vivasol,® CMC (croscarmellose sodium), a superdistintegrant available from JRS Pharma, as set forth in Table 20.

TABLE 20

| Ingredient | % of formulation |
|---|---|
| MCC | 23 |
| CSD | 2 |
| Vivasol CMC | 5 |
| Total | 30 |
| Dry added | 70 |
| fructose:mannitol 1:1 | |
| Powder Total | 100 |
| Solids content of MCC | 17.6 |
| slurry - sulfatate | |
| Required weight of MCC | 1.96 |
| slurry (kg) | |
| MCC solids target | 9.2 |
| Required water added (kg) | 1.79 |
| Total water weight (kg) | 3.41 |
| Total slurry weight (kg) | 3.73 |
| Overall slurry solids target | 12 |

Mixing the CMC into the mixture was easily performed. The mixture is spray-dried at 200 degrees Celsius inlet and 100 degrees Celsius outlet at 55 Hz with the polyol/fructose mixture dry added. The damper was set to one up from full open position. A small 2.5″ dry addition gap was used. Particle size target was 65 μM. The run was successful. The material was bagged.

### Example 13

In Example 13, the process of Example 10 was repeated, however crospovidone XL (100-130μ) was replaced with crospovidone XL-10 having a smaller particle size (30-50μ). The crospovidone XL-10 dispersed well into the slurry. The drier ran successfully and the material was bagged and collected for further analysis.

### Example 14

In Example 14, the formulations of Examples 8, 9, 10, and 12 and 13 were each blended with 0.5% 20 mesh screened PRUV using a Turbula high shear mixer, 500 mg lozenge tablets were made at three target hardnesses: 2, 4 and 6 kp at 25 rpm. When hardness was achieved, tablets were tested for hardness and disintegration as set forth in Example 11 above. The results are set forth below in Tables 21 and 22. The blend using the material from Example 9 did not tablet.

TABLE 21

| | | | |
|---|---|---|---|
| | Hardness | Thickness | Diameter |
| Tablet # | (kP) | (mm) | (mm) |

Example 8 with 0.5% PRUV
Compression force 7.143

| | | | |
|---|---|---|---|
| 1 | 2.24 | 3.93 | 12.65 |
| 2 | 2.65 | 3.91 | 12.65 |
| 3 | 2.55 | 3.92 | 12.63 |
| 4 | 2.75 | 3.91 | 12.64 |
| 5 | 2.75 | 3.91 | 12.64 |
| 6 | 2.14 | 3.93 | 12.64 |
| 7 | 2.45 | 3.9 | 12.65 |
| 8 | 2.04 | 3.89 | 12.65 |
| 9 | 2.34 | 3.92 | 12.65 |
| 10 | 2.55 | 3.92 | 12.65 |
| Average | 2.45 | 3.91 | 12.65 |
| Std. | 0.25 | 0.01 | 0.01 |
| Deviation | | | |

Example 8 with 0.5% PRUV
Compression force 9.857

| | | | |
|---|---|---|---|
| 1 | 3.87 | 3.86 | 12.67 |
| 2 | 3.87 | 3.84 | 12.65 |
| 3 | 4.18 | 3.83 | 12.65 |
| 4 | 3.67 | 3.82 | 12.65 |
| 5 | 3.57 | 3.82 | 12.66 |
| 6 | 3.67 | 3.83 | 12.66 |
| 7 | 3.98 | 3.82 | 12.65 |
| 8 | 3.77 | 3.82 | 12.65 |
| 9 | 3.77 | 3.82 | 12.65 |
| 10 | 4.28 | 3.83 | 12.65 |
| Average | 3.86 | 3.83 | 12.65 |
| Std. | 0.23 | 0.01 | 0.01 |
| Deviation | | | |

Example 8 with 0.5% PRUV
Compression force 11.019

| | | | |
|---|---|---|---|
| 1 | 5.71 | 3.71 | 12.65 |
| 2 | 5.81 | 3.68 | 12.65 |
| 3 | 5.71 | 3.71 | 12.65 |
| 4 | 5.71 | 3.68 | 12.65 |
| 5 | 5.91 | 3.68 | 12.64 |
| 6 | 6.22 | 3.69 | 12.65 |
| 7 | 6.01 | 3.71 | 12.64 |
| 8 | 5.4 | 3.67 | 12.65 |
| 9 | 5.91 | 3.69 | 12.64 |
| 10 | 5.5 | 3.69 | 12.66 |
| Average | 5.79 | 3.69 | 12.65 |
| Std. | 0.24 | 0.01 | 0.01 |
| Deviation | | | |

Example 10 with 0.5% PRUV
Compression force 7.554

| | | | |
|---|---|---|---|
| 1 | 2.04 | 4.02 | 12.64 |
| 2 | 2.24 | 4.04 | 12.64 |
| 3 | 2.24 | 4 | 12.65 |
| 4 | 2.04 | 4.03 | 12.64 |
| 5 | 1.94 | 3.99 | 12.64 |
| 6 | 2.65 | 4 | 12.64 |
| 7 | 2.14 | 4.01 | 12.66 |
| 8 | 1.83 | 4.03 | 12.63 |
| 9 | 2.14 | 4.01 | 12.64 |
| 10 | 1.94 | 4.01 | 12.62 |
| Average | 2.12 | 4.01 | 12.64 |
| Std. | 0.23 | 0.02 | 0.01 |
| Deviation | | | |

Example 10 with 0.5% PRUV
Compression force 11.677

| | | | |
|---|---|---|---|
| 1 | 4.99 | 0.08 | 12.65 |
| 2 | 5.2 | 3.83 | 12.65 |
| 3 | 5.3 | 3.82 | 12.65 |
| 4 | 4.79 | 3.82 | 12.68 |
| 5 | 4.69 | 3.83 | 12.65 |
| 6 | 5.3 | 3.83 | 12.64 |
| 7 | 4.99 | 3.85 | 21.64 |
| 8 | 4.99 | 3.83 | 12.65 |
| 9 | 5.71 | 3.82 | 12.66 |
| 10 | 4.69 | 3.82 | 12.65 |
| Average | 5.07 | 3.45 | 13.55 |
| Std. | 0.32 | 1.19 | 2.84 |
| Deviation | | | |

Example 10 with 0.5% PRUV
Compression force 12.68

| | | | |
|---|---|---|---|
| 1 | 6.22 | 3.81 | 12.66 |
| 2 | 6.32 | 3.78 | 12.65 |
| 3 | 5.71 | 3.78 | 12.65 |
| 4 | 5.4 | 3.78 | 12.65 |
| 5 | 5.5 | 3.79 | 12.64 |
| 6 | 6.63 | 3.78 | 12.65 |
| 7 | 6.01 | 3.78 | 12.65 |
| 8 | 5.81 | 3.76 | 12.65 |
| 9 | 5.4 | 3.77 | 12.64 |
| 10 | 6.12 | 3.8 | 12.65 |
| Average | 5.91 | 3.78 | 12.65 |
| Std. | 0.42 | 0.01 | 0.01 |
| Deviation | | | |

Example 12 with 0.5% PRUV
Compression force 9.286

| | | | |
|---|---|---|---|
| 1 | 2.85 | 3.97 | 12.66 |
| 2 | 3.16 | 3.99 | 12.67 |
| 3 | 2.85 | 3.96 | 12.66 |
| 4 | 3.06 | 3.99 | 12.66 |
| 5 | 2.96 | 3.95 | 12.67 |
| 6 | 2.04 | 3.94 | 12.65 |
| 7 | 2.85 | 3.96 | 12.66 |
| 8 | 3.06 | 3.99 | 12.67 |
| 9 | 2.85 | 3.96 | 12.67 |
| 10 | 2.85 | 3.97 | 12.66 |
| Average | 2.85 | 3.97 | 12.66 |
| Std. | 0.31 | 0.02 | 0.01 |
| Deviation | | | |

Example 12 with 0.5% PRUV
Compression force 11.390

| | | | |
|---|---|---|---|
| 1 | 4.38 | 3.91 | 12.66 |
| 2 | 4.38 | 3.87 | 12.66 |
| 3 | 4.69 | 3.88 | 12.68 |
| 4 | 4.18 | 3.87 | 12.67 |
| 5 | 4.18 | 3.87 | 12.67 |
| 6 | 4.38 | 3.88 | 12.67 |
| 7 | 4.28 | 3.9 | 12.68 |
| 8 | 4.18 | 3.88 | 12.65 |
| 9 | 3.87 | 3.88 | 12.67 |
| 10 | 4.08 | 3.88 | 12.66 |
| Average | 4.26 | 3.88 | 12.67 |
| Std. | 0.22 | 0.01 | 0.01 |
| Deviation | | | |

Example 12 with 0.5% PRUV
Compression force 13.380

| | | | |
|---|---|---|---|
| 1 | 5.4 | 3.64 | 12.65 |
| 2 | 5.91 | 3.74 | 12.65 |
| 3 | 5.2 | 3.72 | 12.66 |
| 4 | 5.81 | 3.74 | 12.65 |
| 5 | 5.71 | 3.74 | 12.67 |
| 6 | 6.12 | 3.75 | 12.66 |
| 7 | 5.3 | 3.74 | 12.66 |
| 8 | 5.5 | 3.75 | 12.66 |
| 9 | 5.61 | 3.77 | 12.68 |
| 10 | 5.62 | 3.73 | 12.66 |
| Average | 5.62 | 3.73 | 12.68 |
| Std. | 0.28 | 0.03 | 0.01 |
| Deviation | | | |

Example 13 with 0.5% PRUV
Compression force 6.045

| | | | |
|---|---|---|---|
| 1 | 1.53 | 4.05 | 12.62 |
| 2 | 1.63 | 4.05 | 12.64 |
| 3 | 1.94 | 4.07 | 12.63 |
| 4 | 1.53 | 4.04 | 12.62 |
| 5 | 1.73 | 4.06 | 12.64 |
| 6 | 1.73 | 4.03 | 12.63 |
| 7 | 1.73 | 4.06 | 12.64 |
| 8 | 1.22 | 4.07 | 12.62 |
| 9 | 1.53 | 4.05 | 12.62 |
| 10 | 1.73 | 4.07 | 12.64 |
| Average | 1.63 | 4.06 | 12.63 |
| Std. | 0.19 | 0.01 | 0.01 |
| Deviation | | | |

Example 13 with 0.5% PRUV
Compression force 10.833

| | | | |
|---|---|---|---|
| 1 | 4.08 | 3.87 | 12.65 |
| 2 | 5.61 | 3.92 | 12.64 |
| 3 | 4.49 | 3.89 | 12.65 |
| 4 | 4.59 | 3.88 | 12.65 |
| 5 | 4.49 | 3.87 | 12.66 |
| 6 | 4.38 | 3.88 | 12.66 |
| 7 | 4.18 | 3.88 | 12.65 |
| 8 | 4.29 | 3.87 | 12.65 |
| 9 | 4.59 | 3.89 | 12.65 |
| 10 | 5.1 | 3.88 | 12.66 |
| Average | 4.58 | 3.88 | 12.65 |
| Std. | 0.46 | 0.01 | 0.01 |
| Deviation | | | |

Example 13 with 0.5% PRUV
Compression force 13.272

| | | | |
|---|---|---|---|
| 1 | 6.52 | 3.83 | 12.66 |
| 2 | 7.24 | 3.84 | 12.65 |
| 3 | 6.83 | 3.83 | 12.66 |
| 4 | 7.24 | 3.84 | 12.65 |
| 5 | 6.83 | 3.85 | 12.65 |
| 6 | 2.34 | 3.84 | 12.34 |
| 7 | 7.14 | 3.84 | 12.644 |
| 8 | 6.42 | 3.82 | 12.65 |
| 9 | 7.44 | 3.84 | 12.66 |
| 10 | 6.93 | 3.84 | 12.64 |
| Average | 6.93 | 3.84 | 12.65 |
| Std. | 0.36 | 0.01 | 0.01 |
| Deviation | | | |

After compaction, tablets were tested for disintegration at 40% relative humidity and 21.6° C. ambient temperature. The results are set forth in Table 22 below for blends made from Example 9, 11, 13 and 14 at each compaction force set out in Table 21 above.

TABLE 22

| | Type | | | | | | | | | | | | |
|---|---|---|---|---|---|---|---|---|---|---|---|---|---|
| | ½″ | | | 10 tab | | | | | | | | | Average |
| | lozenge | Average | Std. | wt | Average | Std | Disintegration time | | | | | | Disintegration |
| Sample | (L) | Kp | Deviation | (mg) | kN | Deviation | (sec) | | | | | | time (sec) |
| Example | L | 2.45 | 0.25 | 4991.7 | 7.143 | 0.331 | 5 | 31 | 7 | 11 | 11 | 7 | 12 |
| 8 blend | | | | | | | | | | | | | |
| Example | L | 3.86 | 0.23 | 5058.4 | 9.857 | 0.212 | 3 | 11 | 7 | 9 | 7 | 7 | 7 |
| 8 blend | | | | | | | | | | | | | |
| Example | L | 5.79 | 0.24 | 4993.3 | 11.019 | 0.333 | 5 | 19 | 9 | 7 | 9 | 7 | 9 |
| 8 blend | | | | | | | | | | | | | |
| Example | L | 2.12 | 0.23 | 4949.1 | 7.554 | 0.251 | 3 | 5 | 1 | 11 | 5 | 3 | 5 |
| 10 blend | | | | | | | | | | | | | |
| Example | L | 5.07 | 0.32 | 4957.6 | 11.677 | 0.398 | 5 | 7 | 3 | 3 | 7 | 7 | 5 |
| 10 blend | | | | | | | | | | | | | |
| Example | L | 5.91 | 0.42 | 4949.9 | 12.68 | 0.394 | 5 | 11 | 3 | 5 | 7 | 7 | 6 |
| 10 blend | | | | | | | | | | | | | |
| Example | L | 2.85 | 0.31 | 5029.9 | 9.286 | 0.503 | 11 | 15 | 4 | 9 | 13 | 7 | 10 |
| 12 blend | | | | | | | | | | | | | |
| Example | L | 4.26 | 0.22 | 5060.7 | 11.39 | 0.403 | 17 | 17 | 9 | 11 | 13 | 9 | 13 |
| 12 blend | | | | | | | | | | | | | |
| Example | L | 5.62 | 0.28 | 4926.5 | 13.38 | 0.393 | 13 | 11 | 13 | 5 | 9 | 9 | 10 |
| 12 blend | | | | | | | | | | | | | |
| Example | L | 1.63 | 0.19 | 4883.8 | 8.046 | 0.429 | 5 | 3 | 3 | 7 | 3 | 5 | 4 |
| 13 blend | | | | | | | | | | | | | |
| Example | L | 4.58 | 0.46 | 4923.8 | 10.833 | 0.339 | 9 | 17 | 9 | 25 | 13 | 11 | 14 |
| 13 blend | | | | | | | | | | | | | |
| Example | L | 6.93 | 0.36 | 5033.6 | 13.272 | 0.606 | 15 | 15 | 9 | 18 | 13 | 11 | 14 |
| 13 blend | | | | | | | | | | | | | |

### Example 15

In Example 15, a modification of dry feed apparatus from Example 13, with an attachment having 6 exit tubes was tested. The formulation of Example 13 was employed during the experiment to provide a successful run.

### Example 16

In Example 16, the dry powder feed rate determination for 1:1 fructose:mannitol powder was repeated according to the process set forth in Example 11 above with an agitator rate of 350. The results are set out in Table 23 below.

TABLE 23

| | | | | g/m |
|---|---|---|---|---|
| | | | | adjusted |
| | | Trial Time | | for 0.5% |
| Feed Rate | Grams | min | g/m | water |
| 125 | 5.6 | 2 | 2.8 | 2.79 |
| 175 | 44.6 | 2 | 22.3 | 22.19 |
| 225 | 79 | 2 | 39.5 | 39.30 |
| 275 | 117.2 | 2 | 58.6 | 58.31 |
| 325 | 160.5 | 2 | 80.25 | 79.85 |

Using the equation from the linear trend line (y=0.3805x−45.123 R²=0.9986), values for feed rate were obtained to match desired output to solids in the slurry.

### Example 17

In Example 17, a 1:1 blend of mannitol and fructose (Krystar 300) was blended as set forth in the above examples. The polyol/fructose blend was then used in a spray drying run as set forth in Example 10 and the lady finger dry powder addition attachment was used. Based on the trend line found on 14.9% solids slurry chart as set forth above, a slurry feed rate chart was prepared using the formula y=32.34x−25.681. No clogging was reported in the feed tube and the material was collected for further processing.

### Example 18

In Example 18, another formulation as set forth in Example 17 was prepared except that the formulation did not contain CSD as set forth in Table 24.

TABLE 24

| Ingredient | % of formulation |
|---|---|
| MCC | 25 |
| Crospovidone XL | 5 |
| Total | 30 |
| Dry added | 70 |
| fructose:mannitol 1:1 | |
| Powder Total | 100 |
| Solids content of MCC | 17.6 |
| slurry - sulfatate | |
| Required weight of MCC | 2.13 |
| slurry (kg) | |
| MCC solids target | 10% |
| Required water added (kg) | 1.62 |
| Total water weight (kg) | 3.38 |
| Total slurry weight (kg) | 3.75 |
| Overall slurry solids target | 12 |

This run was also successful. The material was bagged for further processing.

An analysis of particle size revealed that the material from Example 18 produced a more uniform particle size compared to earlier created materials discussed above.

### Example 19

In Example 19, the material from Examples 17 and 18 were each blended with 0.5% 20 mesh screened PRUV in a high shear mixer for 5 minutes and subsequently compressed into tablets as set forth in Example 10 using L tooling. The tablets were then tested as set forth in Example 10 for compaction, hardness and disintegration at 22% humidity and 22 degrees Celsius. The results are set forth in Table 25 and 26.

TABLE 25

| Material | | | | | | | | | | | | | | |
|---|---|---|---|---|---|---|---|---|---|---|---|---|---|---|
| blended | | | | | | | | | | | | | | Average |
| with | | | | | | Ejection | | | | | | | | Disintegration |
| PRUV | 10 tab wt | Average | Std. | Average | Std | Force | Std | Disintegration time | | | | | | time |
| 0.5% | (mg) | Kp | Dev. | kN | Dev. | kN | Dev. | (sec) | | | | | | (sec) |
| Ex. 17 | 4960.9 | 2.05 | 0.18 | 7.750 | 0.127 | 72.09 | 2.726 | 21 | 13 | 9 | 5 | 9 | 3 | 10 |
| Ex. 17 | 6270.1 | 7.52 | 1 | 15.32 | 0.47 | 163.8 | 6.452 | 41 | 21 | 29 | 19 | 25 | 7 | 24 |
| Ex. 18 | 5069.3 | 1.87 | 0.13 | 9.1 | 0.213 | 74.13 | 2.676 | 5 | 5 | 7 | 5 | 7 | 0 | 6 |
| Ex. 18 | 6335.5 | 7.86 | 0.67 | 18.17 | 0.255 | 154.5 | 3.416 | 15 | 15 | 21 | 15 | 15 | 0 | 16 |

TABLE 26

| | | |
|---|---|---|
| Hardness | Thickness | Diameter |
| (kP) | (mm) | (mm) |

Example 17 with 0.5% PRUV
Compression force 7.75

| | | | |
|---|---|---|---|
| Average | 2.05 | 4.09 | 12.63 |
| Std. | 0.18 | 0.02 | 0.01 |
| Deviation | | | |

Example 17 with 0.5% PRUV
Compression force 15.32

| | | | |
|---|---|---|---|
| Average | 7.52 | 4.65 | 12.63 |
| Std. | 1.00 | 0.02 | 0.01 |
| Deviation | | | |

Example 18 with 0.5% PRUV
Compression force 9.1

| | | | |
|---|---|---|---|
| Average | 1.87 | 4.1 | 12.63 |
| Std. | 0.13 | 0.01 | 0.01 |
| Deviation | | | |

Example 18 with 0.5% PRUV
Compression force 18.17

| | | | |
|---|---|---|---|
| Average | 7.86 | 4.53 | 12.62 |
| Std. | 0.67 | 0.02 | 0.01 |
| Deviation | | | |

The data suggest that much more force is needed to make a tablet of comparable hardness with the material from Example 18 (i.e., without CSD).

### Example 20

In Example 20, four blends as set forth in Table 27 were prepared, compacted and tested for hardness and disintegration. The blends include the material from Examples 17 and Example 18, a commercially available BASF product Ludiflash® (containing mannitol, crospovidone and polyvinyl acetate), and a dry blended mixture which has not been coprocessed.

TABLE 27
Example 17

| | | | | | | Blend |
|---|---|---|---|---|---|---|
| | | | | | | required |
| | | Blending | | | | amount |
| Component | Formulation | Series | | Tablet (mg) | Percent | (g) |
| ODT | Ex. 17 | 1 | | 588 | 98 | 98 |
| | material | | | | | |
| Lubricant | PRUV | 1 | | 12 | 2 | 2 |
| | | | Target | 600 | 100 | 100 |
| | | | Total | | | |
| | | | Total | 600 | 100 | 100 |

Blending Schedule

| Series | Components | Time |
|---|---|---|
| 1 | ODT, | 5 |
| | lubricant | |

Example 18

| | | | | | | Blend |
|---|---|---|---|---|---|---|
| | | | | | | required |
| | | Blending | | Tablet | | amount |
| Component | Formulation | Series | | (mg) | Percent | (g) |
| ODT | Ex. 18 | 1 | | 588 | 98 | 98 |
| | material | | | | | |
| Lubricant | PRUV | 1 | | 12 | 2 | 2 |
| | | | Target | 600 | 100 | 100 |
| | | | Total | | | |
| | | | Total | 600 | 100 | 100 |

Blending Schedule

| Series | Components | Time |
|---|---|---|
| 1 | ODT, | 5 |
| | lubricant | |

BASF Ludiflash

| | | | | | | Blend |
|---|---|---|---|---|---|---|
| | | | | | | required |
| | | Blending | | Tablet | | amount |
| Component | Formulation | Series | | (mg) | Percent | (g) |
| ODT | Ludiflash | 1 | | 588 | 98 | 98 |
| Lubricant | PRUV | 1 | | 12 | 2 | 2 |
| | | | Target | 600 | 100 | 100 |
| | | | Total | | | |
| | | | Total | 600 | 100 | 100 |

Blending Schedule

| Series | Components | Time |
|---|---|---|
| 1 | ODT, | 5 |
| | lubricant | |

ODT Matrix Dry Blend HD50 with CSD

| | | | | | Blend |
|---|---|---|---|---|---|
| | | | | | required |
| | | Blending | Tablet | | amount |
| Component | Formulation | Series | (mg) | Percent | (g) |
| Binder | Emcocel | 2 | | 23 | 230 |
| | HD50 | | | | |
| Glidant | CSD | 1 | | 2 | 20 |
| Sugar 1 | Mannitol | 1 | | 35 | 350 |
| | Pearlitol 50C | | | | |
| Sugar 2 | Krystar 300 | 1 | | 35 | 350 |
| Disintegrant | Crospovidone | 2 | | 5 | 50 |
| | XL | | | | |
| | | | | 100 | 100 |
| | | | | 100 | 100 |
| Prosolv dry | ODT matrix | | 588 | 98 | 98 |
| blend | dry blend | | | | |
| Lubricant | PRUV | | 12 | 2 | 2 |
| | | | 600 | | |
| | | | 600 | 100 | 100 |

Blending Schedule

| Series | Components | Time |
|---|---|---|
| 1 | Prosolv | 5 |
| | HD50/sugars | |
| 2 | Disintegrant | 5 |
| 3 | Lubricant | 5 |

The compression, hardness and disintegration results are set forth in Table 28 below. Tooling type L was employed.

TABLE 28

| Material | | | | | | | | | | | | |
|---|---|---|---|---|---|---|---|---|---|---|---|---|
| blended | | | | | | | | | | | | |
| with | | | 10 tab | | | | | | | | | Average |
| PRUV | Average | Std. | wt | Average | Std | Disintegration time | | | | | | Disintegration |
| 0.5% | Kp | Deviation | (mg) | kN | Deviation | (sec) | | | | | | time (sec) |
| Ex. 17 | 1.91 | 0.26 | 6079.7 | 6.898 | 0.128 | 46 | 40 | 38 | 42 | 52 | 48 | 46 |
| Ex. 17 | 3.18 | 0.54 | 6097.4 | 10.543 | 0.169 | 42 | 38 | 42 | 40 | 42 | 42 | 41 |
| Ex. 17 | 5.79 | 0.24 | 6087.7 | 15.172 | 0.291 | 50 | 56 | 98 | 50 | 46 | 50 | 50 |
| Ex. 18 | 3.47 | 5.08 | 6064.3 | 9.479 | 0.325 | 52 | 58 | 90 | 56 | 44 | 44 | 49 |
| Ex. 18 | 4.02 | 0.4 | 6069.4 | 12.029 | 484 | 44 | 42 | 36 | 50 | 42 | 42 | 48 |
| Ex. 18 | 5.92 | 0.36 | 5994.1 | 19.259 | 0.221 | 58 | 60 | 106 | 58 | 54 | 54 | 56 |
| ODT dry | 1.66 | 0.11 | 6033.4 | 9.504 | 0.295 | 58 | 58 | 96 | 54 | 48 | 48 | 53 |
| blend | | | | | | | | | | | | |
| ODT dry | 3.28 | 0.34 | 5891.4 | 17.265 | 0.337 | 42 | 44 | 68 | 38 | 36 | 36 | 39 |
| blend | | | | | | | | | | | | |
| ODT dry | 3.26 | 0.16 | 602534 | 22.436 | 0.449 | 46 | 50 | 46 | 46 | 56 | 56 | 50 |
| Blend | | | | | | | | | | | | |
| Ludiflash | 2.25 | 0.24 | 6017.5 | 6.046 | 0.047 | 38 | 40 | 34 | 44 | 54 | 42 | 44 |
| Ludiflash | 4.14 | 0.12 | 5990.3 | 10.833 | 0.082 | 50 | 30 | 86 | 48 | 42 | 44 | 47 |
| Ludiflash | 6.55 | 0.17 | 6011.2 | 13.272 | 0.099 | 42 | 42 | 32 | 46 | 48 | 42 | 44 |

### Example 21

In Example 21, three blends for compaction and disintegration were prepared including 21-1: the formula from Example 17 utilizing the attachment set forth in Example 15, and a rotary atomizer, 21-2: the formula of Example 17 utilizing a nozzle atomizer (slurry pump and air pump) as the pilot scale-up batch, and 21-3: a custom Prosolv 7% CSD dry blend formulation without coprocessing as set forth in Table 29 below.

TABLE 29

| | | | | | Blend |
|---|---|---|---|---|---|
| | | | | | required |
| | | Blending | Tablet | | amount |
| Component | Formulation | Series | (mg) | Percent | (g) |
| Binder | Prosolv | 1 | | 23 | 115 |
| | custom 7% | | | | |
| | CSD | | | | |
| Sugar 1 | Mannitol - | 1 | | 35 | 175 |
| | pearlitol 50C | | | | |
| Sugar 2 | Fructose | 1 | | 35 | 175 |
| | Krystar 300 | | | | |
| Disintegrant | Crospovidone | 1 | | 5 | 25 |
| | xl | | | | |
| | | | | 100 | 500 |
| | | | | 98 | 490 |
| Prosolv dry | ODT matrix | 2 | 588 | 98 | 245 |
| blend | dry blend | | | | |
| Lubricant | PRUV | 2 | 12 | 2 | 5 |
| | | Target | 600 | | 250 |
| | | total | | | |
| | | Total | 600 | 100 | 250 |

Blending Schedule

| Series | Components | Time |
|---|---|---|
| 1 | Prosolv | 5 |
| | HD50/sugars/ | |
| | Disintegrant | |
| 2 | Lubricant | 5 |

The results of compaction, hardness and disintegration are set forth in Table 30 below. It was also determined that 21-2 had a density of about 0.5 to about 0.55 g/ml whereas 21-1 had a density of about 0.75 g/ml. For purposes of compressing an ODT, 21-2 had better workability.

TABLE 30

| | | | 10 tab | | | Disintegration | | | | | | |
|---|---|---|---|---|---|---|---|---|---|---|---|---|
| | Average | Std | weight | Average | Std | Time (sec) | | | | | | |
| Sample | kp | Deviation | (mg) | kN | Deviation | 1 | 2 | 3 | 4 | 5 | 6 | 7 |
| 21.1 | 1.57 | 0.21 | 6048.8 | 5.680 | 0.185 | 32 | 28 | 26 | 28 | 30 | 36 | 28 |
| 21.1 | 3.13 | 0.29 | 5943.3 | 8.940 | 0.075 | 30 | 30 | 26 | 28 | 34 | 28 | 22 |
| 21.1 | 4.47 | 0.30 | 5944.3 | 10.980 | 0.182 | 24 | 16 | 30 | 30 | 30 | 24 | 48 |
| 21.1 | 7.31 | 0.20 | 6057.3 | 15.950 | 0.252 | 32 | 32 | 30 | 30 | 28 | 30 | 26 |
| 21.2 | 1.64 | 0.13 | 6029.4 | 3.760 | 0.113 | 58 | 46 | 62 | 56 | 60 | 60 | 106 |
| 21.2 | 3.41 | 0.17 | 6042.3 | 5.700 | 0.089 | 42 | 56 | 84 | 74 | 52 | 44 | 40 |
| 21.2 | 6.01 | 0.30 | 6103.6 | 8.150 | 0.224 | 128 | 108 | 122 | 102 | 104 | 104 | 92 |
| 21.2 | 7.62 | 0.43 | 5995.0 | 9.570 | 0.188 | 258 | 216 | 224 | 230 | 220 | 220 | 214 |
| 21.3 | 1.56 | 0.09 | 6022.5 | 5.990 | 0.137 | 30 | 26 | 28 | 28 | 24 | 26 | 22 |
| 21.3 | 2.69 | 0.27 | 5920.8 | 8.570 | 0.112 | 30 | 30 | 26 | 26 | 38 | 32 | 16 |
| 21.3 | 4.25 | 0.16 | 6021.2 | 11.450 | 0.253 | 30 | 28 | 30 | 30 | 30 | 32 | 20 |
| 21.3 | 5.93 | 0.23 | 6015.3 | 14.900 | 0.249 | 22 | 22 | 22 | 20 | 24 | 32 | 14 |
| 21.3 | 7.15 | 0.36 | 5911.2 | 18.100 | 0.467 | 24 | 22 | 24 | 24 | 24 | 26 | 22 |

| | | | | | | | | | | | | Avg. |
|---|---|---|---|---|---|---|---|---|---|---|---|---|
| | Disintegration | | | | | | | | | | | Dis. |
| | Time (sec) | | | | | | | | | | | Time |
| Sample | 8 | 9 | 10 | 11 | 12 | 13 | 14 | 15 | 16 | 17 | 18 | (sec) |
| 21.1 | 28 | 26 | 26 | 28 | 26 | 34 | 34 | 34 | 32 | 32 | 30 | 30 |
| 21.1 | 26 | 22 | 24 | 24 | 26 | 22 | 24 | 26 | 28 | 28 | 24 | 26 |
| 21.1 | 32 | 30 | 28 | 30 | 32 | 30 | 34 | 32 | 32 | 34 | 34 | 31 |
| 21.1 | 50 | 40 | 46 | 44 | 54 | 36 | 50 | 42 | 44 | 50 | 60 | 40 |
| 21.2 | 52 | 66 | 80 | 60 | 76 | 68 | 62 | 42 | 62 | 70 | 60 | 64 |
| 21.2 | 66 | 60 | 30 | 70 | 74 | 38 | 86 | 110 | 68 | 112 | 86 | 66 |
| 21.2 | 88 | 96 | 98 | 108 | 88 | 100 | 116 | 108 | 132 | 116 | 110 | 107 |
| 21.2 | 192 | 208 | 202 | 186 | 214 | 194 | 200 | 188 | 180 | 224 | 204 | 210 |
| 21.3 | 20 | 20 | 20 | 18 | 20 | 14 | 18 | 16 | 14 | 14 | 14 | 21 |
| 21.3 | 16 | 18 | 16 | 16 | 22 | 22 | 20 | 18 | 20 | 20 | 22 | 23 |
| 21.3 | 18 | 18 | 18 | 16 | 22 | 18 | 16 | 18 | 16 | 20 | 24 | 22 |
| 21.3 | 12 | 16 | 12 | 14 | 18 | 16 | 14 | 14 | 14 | 16 | 18 | 18 |
| 21.3 | 20 | 20 | 18 | 18 | 18 | 18 | 22 | 22 | 18 | 18 | 22 | 21 |

Dilution potential of the 21-2 was studied by preparing 4 blends all having 0.5% PRUV and 0%, 10%, 20%, 30% and 40% ascorbic acid respectively, prepared according to the procedure set forth in Example 17. The blends were then tested for compaction, hardness and disintegration. The results are set forth in Table 31 below.

TABLE 31

| Sample | | | | | | | | | | | | |
|---|---|---|---|---|---|---|---|---|---|---|---|---|
| amount | | | 10 tab | | | | | | | | | Average |
| ascorbic | Average | Std | weight | Average | Std | Disintegration Time | | | | | | Disintegration |
| acid | kp | Deviation | (mg) | kN | Deviation | (sec) | | | | | | Time (sec) |
| 0% | 2.26 | 0.13 | 6011.0 | 3.997 | 0.059 | 60 | 56 | 72 | 52 | 62 | 68 | 62 |
| 0% | 3.50 | 0.13 | 6003.0 | 5.969 | 0.056 | 76 | 50 | 78 | 74 | 32 | 70 | 63 |
| 0% | 5.64 | 0.25 | 5926.0 | 8.086 | 0.126 | 102 | 186 | 88 | 194 | 210 | 200 | 163 |
| 0% | 7.75 | 0.50 | 5967.0 | 9.741 | 0.271 | 124 | 312 | 154 | 296 | 344 | 348 | 263 |
| 10% | 1.70 | 0.14 | 6015.0 | 4.131 | 0.058 | 46 | 44 | 42 | 50 | 50 | 46 | 46 |
| 10% | 3.55 | 0.26 | 5998.0 | 6.710 | 0.095 | 44 | 66 | 58 | 42 | 44 | 36 | 48 |
| 10% | 5.67 | 0.22 | 5987.0 | 9.933 | 0.180 | 120 | 162 | 188 | 142 | 168 | 122 | 150 |
| 10% | 7.96 | 0.34 | 6052.0 | 12.289 | 0.974 | 174 | 166 | 180 | 176 | 178 | 146 | 170 |
| 20% | 2.00 | 0.17 | 6069.0 | 6.466 | 0.084 | 52 | 54 | 64 | 62 | 52 | 44 | 55 |
| 20% | 3.70 | 0.24 | 6035.0 | 8.846 | 0.128 | 58 | 66 | 64 | 54 | 68 | 42 | 59 |
| 20% | 5.72 | 0.42 | 6014.0 | 12.085 | 0.367 | 70 | 136 | 78 | 68 | 70 | 72 | 82 |
| 20% | 7.87 | 0.40 | 5995.0 | 15.359 | 0.152 | 130 | 132 | 218 | 130 | 294 | 154 | 176 |
| 30% | 2.13 | 0.21 | 6061.0 | 7.439 | 0.147 | 42 | 44 | 52 | 52 | 44 | 46 | 47 |
| 30% | 3.80 | 0.27 | 6088.0 | 11.883 | 0.164 | 26 | 34 | 30 | 70 | 60 | 36 | 43 |
| 30% | 6.00 | 0.32 | 6018.0 | 17.551 | 0.325 | 90 | 94 | 92 | 94 | 76 | 84 | 88 |
| 30% | 6.55 | 0.40 | 6000.0 | 19.502 | 0.492 | 166 | 96 | 86 | 80 | 104 | 88 | 103 |
| 40% | 1.97 | 0.13 | 6090.0 | 9.167 | 0.374 | 36 | 40 | 34 | 54 | 42 | 30 | 39 |
| 40% | 3.27 | 0.13 | 60007.0 | 14.927 | 0.296 | 38 | 34 | 36 | 34 | 32 | 28 | 34 |
| 40% | 4.24 | 0.27 | 6066.0 | 19.608 | 0.162 | 38 | 60 | 40 | 54 | 38 | 34 | 44 |

### Example 22

In Example 22, a 25% ascorbic acid (sourced from Spectrum 100% pure), blend formulation using the ODT matrix 21-2 described above is set forth in Table 32.

TABLE 32

| | Batch Weight |
|---|---|
| Tablet Formulation | Requirement (g) |
| Ascorbic Acid | 62.5 |
| ODT Matrix 31-2 | 185.6 |
| Orange Flavor (Firmenich) | 0.6 |
| Pruv | 1.3 |
| Total | 250 |
| Total # of tablets (90% | 225 |
| recovery) | |
| Tooling type | Lozenge |
| Tooling dimensions | 0.625 |
| Hob # | Natoli 12-07 |

The blend was prepared utilizing a Turbula high shear mixer with 20 mesh screened Pruv as set forth in the above examples. Compaction, hardness and disintegration testing was performed as set forth in the above examples using a TBH-30MD disintegration tester. The powder feeder attachment was set to 5 and ⅝″ L tooling was used with the tablet press at 25 rpm. Tablets of 1000 mg+/−20 mg were made at 2-3 kp. When desired weight and hardness were reached, compaction forces were recorded. The results are set forth below in Table 33.

TABLE 33

| | | | | | | | | | | | Avg. |
|---|---|---|---|---|---|---|---|---|---|---|---|
| Avg. | Std. | 10 tab | Avg. | Std. | Disintegration time (sec) | | | | | | Dis. |
| kp | dev. | weight | kN | Dev. | 1 | 2 | 3 | 4 | 5 | 6 | time |
| 2.25 | 0.13 | 10039 | 11.51 | 0.172 | 64 | 64 | 72 | 62 | 72 | 60 | 66 |

Under in-vivo testing conditions, it was noted that the tablets dissolved in about 20-25 seconds. A tart taste was detected.

### Example 23

In Example 23, a 31% ascorbic acid (sourced from Spectrum 100% pure) with aspartame (0.5%) blend formulation using the ODT matrix 21-2 described above is set forth in Table 34. During this blend, ascorbic acid and aspartame were blended for 10 minutes, then the remaining ingredients were added, and blended for 15 minutes.

TABLE 34

| | Batch Weight |
|---|---|
| Tablet Formulation | Requirement (g) |
| Ascorbic Acid | 62.5 |
| ODT Matrix 21-2 | 183.8 |
| Orange Flavor (Firmenich) | 1.3 |
| Aspartame | 1.3 |
| Pruv | 1.3 |
| Total | 250 |
| Total # of tablets (90% | 225 |
| recovery) | |
| Tooling type | Lozenge |
| Tooling dimensions | 0.625 |
| Hob # | Natoli 12-07 |

When desired weight and hardness were reached, compaction forces were recorded. The results are set forth below in Table 35.

TABLE 35

| | | | | | | | | | | | Avg. |
|---|---|---|---|---|---|---|---|---|---|---|---|
| Avg. | Std. | 10 tab | Avg. | Std. | Disintegration time (sec) | | | | | | Dis. |
| kp | dev. | weight | kN | Dev. | 1 | 2 | 3 | 4 | 5 | 6 | time |
| 2.34 | 0.21 | 10004.5 | 10.05 | 0.26 | 82 | 96 | 80 | 71 | 70 | 50 | 75 |

### Example 24

In Example 24, a 50% ibuprofen (sourced from Spectrum 100% pure) with aspartame (0.5%) blend formulation using the ODT matrix 31-2 described above is set forth in Table 36. The tablets were prepared utilizing a ⅜″ flat faced B tooling during tableting.

TABLE 36

| | Batch Weight |
|---|---|
| Tablet Formulation | Requirement (g) |
| Ibuprofen (Balchem 80%) | 100.3 |
| ODT Matrix 21-2 | 98 |
| Orange Flavor (Firmenich) | 0.7 |
| Pruv 113 | 1 |
| Total | 200 |
| Total # of tablets (75% | 602 |
| recovery) | |
| Tooling type | ⅜″ flat |
| | faced B (RFF) |

When desired weight and hardness were reached, compaction forces were recorded at 46% relative humidity and 20.3 degrees Celsius ambient temperature. The results are set forth below in Table 37.

TABLE 37

| | | | | | | | | | | | Avg. |
|---|---|---|---|---|---|---|---|---|---|---|---|
| Avg. | Std. | 10 tab | Avg. | Std. | Disintegration time (sec) | | | | | | Dis. |
| kp | dev. | weight | kN | Dev. | 1 | 2 | 3 | 4 | 5 | 6 | time |
| 2.69 | 0.2 | 2485.9 | 1.3 | 0.055 | 82 | 96 | 80 | 71 | 70 | 50 | 75 |

### Example 25

In Example 25, one placebo blend of the ODT 21-2 material and one placebo blend with Ludiflash ODT material was prepared according the procedure set forth in the above examples. ⅜″ lozenge tooling was used in the tableting process. Both 250 g batches contained 249.4 g respective ODT material and 0.6 g lubricant. Tableting, compression and hardness results are set forth in Table 38 below.

TABLE 38

| | Avg. | Std. | Avg. | Std. |
|---|---|---|---|---|
| Sample | kp | dev. | kN | Dev. |
| 21-2 | 2.67 | 0.46 | 3.68 | 0.06 |
| 21-2 | 3.4 | 0.6 | 5.03 | 0.14 |
| 21-2 | 6.16 | 0.35 | 6.89 | 0.16 |
| 21-2 | 8.63 | 0.31 | 8.2 | 0.26 |
| Ludiflash | 2.76 | 0.46 | 3.71 | 0.06 |
| Ludiflash | 4.63 | 0.36 | 6.13 | 0.1 |
| Ludiflash | 5.58 | 0.35 | 8.59 | 0.23 |
| Ludiflash | 7.02 | 0.31 | 10.74 | 0.15 |

The data suggest that less force is needed to make harder tablets in the formulations 21-2 ODT blends.

### CONCLUSION

Many other variations of the present invention will be apparent to those skilled in the art and are meant to be within the scope of the claims appended hereto. The foregoing specification alludes to beliefs, hypothesis and conclusions of the inventors based on their experience in the field, the reports of others (such as those identified in the publications identified herein), and experiments conducted and reported herein, and are provided for purposes of (possible) explanation only and are not meant to limit the invention in any manner whatsoever.

The disclosure of all published documents including but not limited to patents recited herein are hereby incorporated by reference in their entireties.

1. A pharmaceutical excipient composition, comprising agglomerated particles of a cellulosic material, a metal oxide, a polyol, a sugar, and a disintegrant.

2. The excipient of claim 1, wherein the cellulosic material is microcrystalline cellulose.

3. The excipient of claim 1, comprising from about 10 to about 40% cellulosic material and from about 1 to about 10% metal oxide.

4. The excipient of claim 2, wherein the metal oxide is a fumed or colloidal metal oxide.

5. The excipient of claim 4, wherein the fumed or colloidal metal oxide is colloidal silicon dioxide.

6. The excipient of claim 3 wherein the cellulosic component and the metal oxide are agglomerated together such that they are in intimate association with each other prior to mixing with the polyol, sugar and disintegrant.

7. The excipient of claim 3 wherein the cellulosic component, the metal oxide and the disintegrant are agglomerated together such that they are in intimate association with each other prior to mixing with the polyol and sugar.

8. The excipient of claim 6, wherein the cellulosic material is microcrystalline cellulose, the silicon dioxide is colloidal silicon dioxide, the disintegrant is crospovidone XL, and the polyol/sugar blend is fructose/mannitol in a 1:1 blend.

9. The excipient of claim 7, wherein the cellulosic material is microcrystalline cellulose, the silicon dioxide is colloidal silicon dioxide, the disintegrant is crospovidone XL, and the polyol/sugar blend is fructose/mannitol in a 1:1 blend.

10. The excipient of claim 1, wherein the polyol is selected from the group consisting of sorbitol, mannitol, xylitol, erythritol, maltitol, lactitol, isomalt, and mixtures thereof.

11. The excipient of claim 10, wherein the sugar is selected from the group consisting of lactose, fructose, dextrose, sucrose, maltose, xylose, mannose, and mixtures thereof.

12. The excipient of claim 11, wherein polyol is mannitol.

13. The excipient of claim 10, wherein the sugar is fructose.

14. The excipient of claim 1, wherein the ratio of the polyol component to the sugar component is from about 99.1:0.9 to about 0.9:99.1.

15. The excipient of claim 1, wherein the ratio of the polyol component to the sugar component is from about 80:20 to about 20:80.

16. The excipient of claim 1, wherein where the ratio of the polyol component to the sugar component is from about 60:40 to about 40:60.

17. The excipient of claim 1, wherein the disintegrant comprises from about Ito about 10% of the composition and is selected from the group consisting of corn starch, modified corn starch, potato starch, modified potato starch, pregelatinized starch, sodium starch glycolate, a cross-linked polyvinyl pyrrolidone, alginate, a cellulosic, an ion exchange resin, a natural gum, a modified natural gum, a synthetic gum, chitin, chitosan, clay, agar, a gas evolving disintegrant.

18. The excipient claim 1, wherein the particles have a d₅₀ value of about 50-160 μm.

19. A process of making the excipient of claim 1, comprising:
(i) dry blending the polyol component and sugar component to create a dry blend,
(ii) preparing an aqueous slurry comprising the cellulosic material, the metal oxide and the disintegrant,
(iii) contacting the aqueous slurry with the dry blend to obtain dry excipient particles comprising the cellulosic material, the metal oxide, the disintegrant and the dry blend and,
(iv) recovering the excipient

20. An oral solid dosage from comprising:
a compressed mixture of an excipient comprising agglomerated particles of a cellulosic material, a compressibility augmenting agent, one or more polyols, one or more sugars, and a disintegrant,
an effective amount of an active agent; and
an optional sweetening agent and an optional flavoring agent,
wherein the oral solid dosage form substantially disintegrates within about 90 seconds when placed on the tongue of a patient.

21. The oral solid dosage form of claim 20, wherein the cellulosic material is microcrystalline cellulose and the compressibility augmenting agent is selected from the group consisting of a metal oxide and a surfactant.

22. A pharmaceutical excipient composition, comprising agglomerated particles of
a cellulosic material in intimate association with a compressibility augmenting agent selected from the group consisting of a metal oxide, a surfactant and a mixture of the foregoing,
a polyol,
a sugar, and
a disintegrant.
